(19) United States
(12) Patent Application Publication
(10) Pub. No.: US 2020/0308610 A1
(43) Pub. Date: Oct. 1, 2020

(54) ALPHA-KETOISOCAPROIC ACID AND & ALPHA-KETO-3-METHYLVALERIC ACID DECARBOXYLASES AND USES THEREOF

(71) Applicant: Aemitis, Inc., Cupertino, CA (US)

(72) Inventors: Goutham N Vemuri, Plymouth, MN (US); Maxim Suvorov, Myersville, MD (US); Olena Lar, Gaithersburg, MD (US); Christopher Snow, Cupertino, CA (US)

(73) Assignee: Aemitis, Inc., Cupertino, CA (US)

(21) Appl. No.: 16/084,136

(22) PCT Filed: Mar. 11, 2017
(86) PCT No.: PCT/US2017/021987

§ 371 Date: Sep. 11, 2018

Related U.S. Application Data
(60) Provisional application No. 62/306,744, filed on Mar. 11, 2016.

(51) Int. Cl. C12P 7/52; C12P 7/16; C12P 7/24

(52) U.S. Cl. CPC C12P 7/52; C12P 7/24; C12P 7/16

(57) ABSTRACT

The disclosure provides α-ketoisocaproic acid decarboxylase and α-keto-3-methylvaleric acid decarboxylase and recombinant microorganisms that host these enzymes. Methods involve the use of recombinant microorganisms to increase the production of isoamyl alcohols, their corresponding acids and their derivatives from carbon sources.

### CROSS-REFERENCE TO RELATED APPLICATIONS

This application claims the benefit of U.S. Provisional Application No. 62/306,744, filed on Mar. 11, 2016, the contents of which is incorporated herein by reference in its entirety.

### FIELD

This disclosure generally relates to microbiology, chemical and biochemical technology. This disclosure also relates to engineered proteins in recombinant microorganisms that are used for producing biochemicals from carbon sources. And this disclosure relates to methods for biological synthesis of biochemicals such as carboxylic acids, alcohols, olefins and their derivatives.

### BACKGROUND

Enzymes are biological catalysts that have the ability to perform the conversion of biological molecules. Traditionally, enzymes are associated with discrete activities (e.g. Koshland D E. The Key-Lock Theory and the Induced Fit Theory. Angew Chem Int Edit. 1994; 33(23-24):2375-8). Established databases such as KEGG and UniProt categorize enzymes into clear-cut annotations and foster an implicit assumption that enzymatic activities are specific. This traditional view of enzyme activity is rapidly changing and enzymes have been found to catalyze additional and completely different types of reactions relative to the natural activity they evolved for. Enzymes that can carry out multiple function (“generalist enzymes”) have been shown to be abundant and play different biological roles than those that have a specific functionality (“specialist enzymes”). The ability of enzymes to fulfill multiple biological roles is called catalytic promiscuity and is believed to have evolved to increase the robustness of cells to stringent conditions (e.g. media switches, starvation, drug/toxin presence, etc.). Promiscuous enzymes have been shown to play an adaptive role and are believed to arise through neutral mutations that are not detrimental to the primary enzymatic activity (Aharoni A, Gaidukov L, Khersonsky O, Mc QGS, Roodveldt C, Tawfik D S. The ‘evolvability’ of promiscuous protein functions. Nat Genet. 2005; 37(1):73-6).

There is some fundamental understanding of the nature of certain amino acid residues (size, charge, polarity, etc.) at specific locations in the structure that impart specific functionality, the precise nature of amino acid makeup that dictate the specificity/promiscuity of enzymes to substrates still largely remains unknown. Enzyme promiscuity has been exploited to “engineer” them to accept or shift their preference to non-natural substrates. In those cases, where there the amino acids that play a critical role in enzyme function are known a priori, site-directed mutagenesis of these specific amino acids has been used successfully. Altering the cofactor specificity of an enzyme is the most successful example of site-directed mutagenesis. In those cases where sufficient information on the structure-function relationship is not known, directed evolution has been the tool of choice for protein engineering. Directed molecular evolution can be used to create proteins such as enzymes with novel functions and properties. Starting with a known natural protein, several rounds of mutagenesis, functional screening, and propagation of successful sequences are performed. The advantage of this process is that it can be used to rapidly evolve any protein without knowledge of its structure. Several different mutagenesis strategies exist, including point mutagenesis by error-prone PCR, cassette mutagenesis, and DNA shuffling.

Branched-chain keto-acid decarboxylases are highly promiscuous enzymes that oxidatively decarboxylate a wide range of α-keto acids such as pyruvate, indolepyruvate and α-keto-3-methyl-valerate, α-ketoisocaproate, etc. into their corresponding aldehydes with the liberation of CO₂ (e.g. J. R. Dickinson, S. J. Harrison and M. J. E. Hewlins 1998. An Investigation of the Metabolism of Valine to Isobutyl Alcohol in Saccharomyces cerevisiae, The Journal of Biological Chemistry, 273:25751-25756; J. R. Dickinson, et al., 1997, A ¹³C nuclear magnetic resonance investigation of the metabolism of leucine to isoamyl alcohol in Saccharomyces cerevisiae, The Journal of Biological Chemistry, 272,26871-26878; J. R. Dickinson, et al., 2003, The Catabolism of Amino Acids to Long Chain and Complex Alcohols in Saccharomyces cerevisiae, The Journal of Biological Chemistry, 278, 8028-8034). Branched-chain keto acids could be formed as a result of the metabolism of branched-chain amino acids such as valine, leucine and isoleucine. Transaminases convert branched-chain amino acids into their corresponding branched-chain keto acids, which are decarboxylated to aldehydes (see FIG. 1). The aldehydes are reduced by alcohol dehydrogenases to form branched-chain alcohols such as isobutanol, 3-methyl-1-butanol, 2-methyl-1-butanol, respectively. More generally, this pathway is known as the Erlich pathway, and is very active in yeasts (Hazelwood, L. A., J. M. Daran, et al. (2008). “The Ehrlich pathway for fusel alcohol production: a century of research on Saccharomyces cerevisiae metabolism.” Appl Environ Microbiol 74(8): 2259-2266.). These alcohols (along with aromatic alcohols derived from amino acids) are called fusel alcohols. The aldehydes can also be oxidized by aldehyde dehydrogenases to form branched-chain organic acids such as isobutyric acid, 3-methylbutyric acid and 2-methylbutyric acid from valine, leucine and isoleucine, respectively. There is an increasing demand for the production of fusel alcohols, in recognition of their importance as biofuels as well as natural flavoring agents and food products. For example, isobutanol is produced using this pathway (Atsumi, S., et al. (2008). “Non-fermentative pathways for synthesis of branched-chain higher alcohols as biofuels.” Nature 451(7174): 86-89; WO2008098227 A2 and citations therein, etc.). Branched-chain organic acids offer a versatile platform for the synthesis of a wide variety of building blocks (see for example, US20140065697). Presently, the branched-chain organic acids 3-methylbutyric acid and 2-methylbutyric acid or their corresponding alcohols (3-methylbutanol and 2-methylbutanol) are produced due to promiscuous action of the decarboxylases that non-specifically act on the parent keto acids: α-Ketoisocaproic acid and α-Keto-3-methylvaleric acid, respectively. There have been attempts to increase the carbon flow into the leucine or isoleucine pathway in order to increase the production of these branched-chain compounds (Connor, et al., (2010), “3-Methyl-1-butanol production in Escherichia coli: random mutagenesis and two-phase fermentation”, Appl Microbiol Biotechnol. 2010 April; 86(4): 1155-1164; Cann and Liao, (2008), “Production of 2-methyl-1-butanol in engineered Escherichia coli”, Appl Microbiol Biotechnol. 2008 November; 81(1):89-98). While these attempts have been moderately successful, the promiscuity of the decarboxylase precluded the targeted production of these branched-chain compounds.

Given the importance of branched-chain alcohols and acids in the food and flavor industry and polymer synthesis, there is an immediate need to develop α-Ketoisocaproic acid decarboxylase and α-Keto-3-methylvaleric acid decarboxylase that can preferentially produce the desired branched-chain compounds such as 3-methylbutyric acid or 3-methylbutanol and 2-methylbutyric acid or 2-methylbutanol, respectively. The present disclosure provides methods and compositions that satisfy this need.

### SUMMARY

The present disclosure provides compositions and methods for producing the branched-chain compounds 3-methylbutyric acid, 3-methylbutanol, 2-methylbutyric acid and/or 2-methylbutanol. Prior to this disclosure, the promiscuity of other decarboxylases such as α-keto isovalerate decarboxylase resulted in the decarboxylation of α-Ketoisocaproic acid or α-Keto-3-methylvaleric acid. As disclosed herein, amino acid sequences have been identified that have high specificity for the decarboxylation of α-Ketoisocaproic acid or α-Keto-3-methylvaleric acid.

The present disclosure provides isolated polynucleotides. Isolated polynucleotides include a nucleotide sequence that encodes for a non-natural polypeptide having α-Ketoisocaproic acid decarboxylase or α-Keto-3-methylvaleric acid decarboxylase activity. In one embodiment, the polypeptide with α-Ketoisocaproic acid decarboxylase activity or α-Keto-3-methylvaleric acid decarboxylase activity is derived from the genera Lactoccus or Kluyeromyces. In further embodiments, the polypeptide with α-Ketoisocaproic acid decarboxylase activity or α-Keto-3-methylvaleric acid decarboxylase activity is derived from the genera Azospirillum, Zymomonas or Saccharomyces. In a specific embodiment, the polypeptide with α-Ketoisocaproic acid decarboxylase activity or α-Keto-3-methylvaleric acid decarboxylase activity is derived from Lactococcus lactis, Kluyveromyces lactis, Azospirillum brasilense, Zymomonas mobilis or Saccharomyces cerevisiae. In yet another embodiment, the amino acid sequence of the polypeptide with α-Ketoisocaproic acid decarboxylase activity or α-Keto-3-methylvaleric acid decarboxylase activity is at least 85% identical to the polypeptide with sequence selected from SEQ ID NOs 1-5.

In some embodiments the specific amino acids are targeted to increase the decarboxylation specificity to α-ketoisocaproic acid or α-keto-3-methylvaleric acid by providing the optimal spacing of the active site pocket and/or by providing hydrogen bonding to stabilize the docking of the desired substrates with the protein molecule. The specific mutations are targeted to perturb the specificity to α-ketoisocaproic acid or α-keto-3-methylvaleric acid or mimic the active site configuration conducive to six-carbon keto acids. In some specific embodiments, α-ketoisocaproic acid decarboxylase activity or α-keto-3-methylvaleric acid decarboxylase activity is enhanced by introducing at least one mutation to the amino acid in SEQ ID NO: 1 at the position corresponding to 110, 461, 377, 286, 538, 542 or 402. In some embodiments, α-ketoisocaproic acid decarboxylase activity or α-keto-3-methylvaleric acid decarboxylase activity is enhanced by introducing at least one mutation to the amino acid in SEQ ID NO: 2 at the position corresponding to 292, 388 or 476. In some embodiments, α-ketoisocaproic acid decarboxylase activity or α-keto-3-methylvaleric acid decarboxylase activity is enhanced by introducing at least one mutation to the amino acid in SEQ ID NO: 3 at the position corresponding to 532, 536, 283, 380, 402 or 461. In some embodiments, α-ketoisocaproic acid decarboxylase activity or α-keto-3-methylvaleric acid decarboxylase activity is enhanced by introducing at least one mutation to the amino acid in SEQ ID NO: 4 at the position corresponding to 290, 388, 392 or 472. In some embodiments, α-ketoisocaproic acid decarboxylase activity or α-keto-3-methylvaleric acid decarboxylase activity is enhanced by introducing at least one mutation to the amino acid in SEQ ID NO: 5 at the position corresponding to 444, 469 or 544.

In some embodiments, α-ketoisocaproic acid decarboxylase activity or α-keto-3-methylvaleric acid decarboxylase activity is enhanced by incorporating at least one of [F110A], [V461A], [V461A, Q377G], [Q377G], [Q377S], [Q3771], [Q377M], [S286Y, M538W, F542V], [G402A] or [F542L] mutations in a polypeptide corresponding to SEQ ID NO: 1. In some embodiments, α-ketoisocaproic acid decarboxylase activity or α-keto-3-methylvaleric acid decarboxylase activity is enhanced by incorporating at least one of [F292S], [F292I], [F292L], [T388A, I476V], [T388A, I476A] or [F292L, T388A, I476V] mutations in a polypeptide corresponding to SEQ ID NO: 2. In some embodiments, α-ketoisocaproic acid decarboxylase activity or α-keto-3-methylvaleric acid decarboxylase activity is enhanced by incorporating at least one of [T283L], [L462E, T283V, F532A, Q536V, M461V], [M461V], [F532V, Q536V], [M380Q, A402G, M461A] or [M380A, A402G, M461A] mutations in a polypeptide corresponding to SEQ ID NO: 3. In some embodiments, α-ketoisocaproic acid decarboxylase activity or α-keto-3-methylvaleric acid decarboxylase activity is enhanced by incorporating at least one of [Y290F, T388S, I472V] or [Y290F, T388S, W393L, I472V] or [T388A, I472V] mutations in a polypeptide corresponding to SEQ ID NO: 4. In some embodiments, α-ketoisocaproic acid decarboxylase activity or α-keto-3-methylvaleric acid decarboxylase activity is enhanced by incorporating [T444Q, L469G, I544V] mutation in a polypeptide corresponding to SEQ ID NO: 5.

The present disclosure also provides methods for enhancing the specificity of the decarboxylase to either α-Ketoisocaproic acid or α-Keto-3-methylvaleric acid. In one embodiment, the amino acid sequence of the non-natural polypeptide having α-Ketoisocaproic acid decarboxylase activity has at least 85% identical to a polypeptide selected from SEQ ID NOs: 18-20. In one embodiment, the amino acid sequence of the non-natural polypeptide having α-Keto-3-methylvaleric acid decarboxylase activity has at least 85% identical to a polypeptide selected from SEQ ID NOs: 21-22.

In some embodiments, the amino acid positions corresponding to the specific amino acids of SEQ ID NO: 1-5 in their homologs, orthologs or paralogs are mutated.

In some embodiments the modified decarboxylases are subjected to further engineering by randomly mutagenizing the amino acids sequences. The random mutagenesis could be carried out by error-prone PCR. In some embodiments, the randomly mutagenized decarboxylase has improved properties than the parent decarboxylase.

In some embodiments, the one or more proteins with non-natural amino acid sequences that have enhanced α-Ketoisocaproic acid decarboxylase activity or α-Keto-3-methylvaleric acid decarboxylase activity have greater than 85%, 90%, 91%, 92%, 93%, 94%, 95%, 96%, 97%, 98% or 99% amino acid sequence identity with SEQ ID NO: 1-5.

In some embodiments, the engineered decarboxylase is accompanied with a dehydrogenase or an oxidoreductase that can reduce 2-methylbutanal or 3-methylbutanal into 2-methylbutanol or 3-methylbutanol, respectively. In some embodiments, the dehydrogenase uses NADH or NADPH as the reducing agent. In some embodiments, the dehydrogenase is an alcohol dehydrogenase with an amino acid sequence at least 65% identical to that of SEQ ID NO: 15 or 16.

In some embodiments, the engineered decarboxylase is accompanied with a dehydrogenase or a oxidoreductase that can oxidize 2-methylbutanal or 3-methylbutanal into 2-methylbutyric acid or 3-methylbutyric acid, respectively. In some embodiments, the dehydrogenase uses NAD⁺ or NADP⁺ as the oxidizing agent. In some embodiments, the dehydrogenase is an aldehyde dehydrogenase with an amino acid sequence at least 65% identical to that of SEQ ID NO: 17.

In some embodiments, nucleotide sequences encoding for the non-natural decarboxylases described herein and encoding for the dehydrogenases are expressed under the control of a promoter in a microorganism that is selected from a eukaryote, bacteria or archaea. The nucleotide sequences are optionally integrated in the genome of the appropriate host organism.

In some embodiments, the branched-chain organic acid or alcohol is produced in a fermenter by the engineered microorganism, and is optionally purified. In some embodiments, the method involves contacting an engineered microorganism with a substrate wherein the microorganism is engineered to produce enzymes in a metabolic pathway that the branched-chain organic acid or alcohol from the substrate. In further embodiments, the method involves culturing the microorganism under conditions whereby branched-chain organic acid or alcohol is produced and harvesting the said product. In some embodiments, the microorganism is further engineered to minimize competing metabolic pathways.

In one aspect, the disclosure provides a polypeptide exhibiting 6-carbon keto acid specific decarboxylase activity comprising a variant of the amino acid sequence of SEQ ID NO: 3, wherein the variant comprises 1-40 amino acid substitutions or deletions when compared to the amino acid sequence of SEQ ID NO: 3.

In another aspect, the disclosure provides a polypeptide exhibiting 6-carbon keto acid specific decarboxylase activity comprising a variant of the amino acid sequence of SEQ ID NO: 1, wherein the variant comprises 1-40 amino acid substitutions or deletions when compared to the amino acid sequence of SEQ ID NO: 1.

In some embodiments of the foregoing aspects, the 6-carbon keto-acid decarboxylase activity of the polypeptide is greater than the 5-carbon keto-acid decarboxylase activity, the 4-carbon keto-acid decarboxylase activity, or the 3-carbon keto-acid decarboxylase activity of the polypeptide. In some embodiments, the 6-carbon keto-acid decarboxylase activity of the polypeptide is greater than the 7-carbon keto-acid decarboxylase activity or the 8-carbon keto-acid decarboxylase activity of the polypeptide. In some embodiments, the 6-carbon keto acid of the polypeptide is α-ketoisocaproic acid.

In some embodiments, the 6-carbon keto acid of the polypeptide is α-keto-3-methylvaleric acid.

In some embodiments, the 1-40 amino acid substitutions or deletion of the polypeptide occurs at a position of SEQ ID NO: 1 selected from the group consisting of F110, V461, Q377, 5286, M538, F542, G402 and F542. In some embodiments, the amino acid substitution of the polypeptide is selected from the group consisting of Ala, Glu, Ser, Met, Tyr, Trp, Val and Leu. In some embodiments, the amino acid substitution of the polypeptide is selected from the group consisting of F110A, V461A, V461A/Q377G, Q377G, Q377S, Q377T, Q377M, S286Y/M538W/F542V, G402A and F542L.

In some embodiments, the 1-40 amino acid substitutions or deletion of the polypeptide occurs at a position of SEQ ID NO: 3 selected from the group consisting of: L462, T283, L384, M380, Q536, M461 and F532. In some embodiments, the amino acid substitution of the polypeptide is selected from the group consisting of: Glu, Ile, Val, Ala, Gln, Met, Leu, Ser, and Phe. In some embodiments, the amino acid substitution of the polypeptide is selected from the group consisting of: F532A, F532M, F532M/Q536F, F532V, F532V/Q536V, L384A, L384F, L384Q, L462E, L462E/T283V/F532A/Q536V/M461V, M380Q, M461A, M461V, Q536A, Q536F, Q536V, T283I, T283L, T283V, T388A/I472V, Y290F/T388S/I472V, and T283V/L384F. In some embodiments of the polypeptide, the amino acid substitution is selected from the group consisting of: F532M/Q536F, F532V, F532V/Q536V, L384A, L462E, L462E/T283V/F532A/Q536V/M461V, M461A, M461V, Q536A, Q536V, and T283L. In some embodiments of the polypeptide, the amino acid substitution or deletion is selected from the group consisting of: F542, Q377, V461, M538, S286, F381 and 1465. In some embodiments of the polypeptide, the amino acid substitution is selected from the group consisting of: Ala, Leu, Gly, and Val. In some embodiments of the polypeptide, the amino acid substitution is selected from the group consisting of: F542A, F542A/V461A, F542L, F542V, F542V/V461A, F542V/V461A/M538V/S286A, I465A, M538A, M538A/S286A, M538L, M538V, M538V/S286V, Q377A, S286A, S286L, S286V, V461A, S286G, F110A, G402A, F542L and V461L. In some embodiments of the polypeptide, the amino acid substitution is selected from the group consisting of: F542V/V461A/M538V/S286A, I465A, M538A and V461L.

In some embodiments of the polypeptide, the variant comprises 1-7 amino acid substitutions or deletions when compared to the amino acid sequence of SEQ ID NO: 3. In some embodiments of the polypeptide, the variant comprises 1-7 amino acid substitutions or deletions when compared to the amino acid sequence of SEQ ID NO: 1. In some embodiments, the polypeptide comprises at least 94% homology with the amino acid sequence of SEQ ID NO: 3. In some embodiments, the polypeptide comprises at least 94% homology with the amino acid sequence of SEQ ID NO: 1.

In another aspect, the disclosure provides methods for producing an aldehyde. The methods include contacting a 6-carbon keto acid with any one of the polypeptides described herein.

In some embodiments of the foregoing methods, the 6-carbon keto acid is α-ketoisocaproic acid. In some embodiments of the foregoing methods, the 6-carbon keto acid is α-keto-3-methylvaleric acid.

In some embodiments, the disclosure provides methods of producing 2-methylbutanol. Such methods include contacting an aldehyde produced by the methods described herein with a dehydrogenase or oxidoreductase. In some embodiments, the disclosure provides methods of producing 3-methylbutanol, including contacting the aldehyde produced by the methods described herein with a dehydrogenase or oxidoreductase. In some embodiments, the dehydrogenase is an alcohol dehydrogenase with at least 75% homology to SEQ ID NOS: 15 or 16.

In still another aspect, the disclosure provides methods of producing 2-methylbutyric acid, including contacting the aldehyde produced by the methods described herein with a dehydrogenase or oxidoreductase. In yet another aspect, the disclosure provides methods of producing 3-methylbutyric acid, including contacting the aldehyde produced by the methods described herein with a dehydrogenase or oxidoreductase. In some embodiments the dehydrogenase is an aldehyde dehydrogenase with at least 75% homology to SEQ ID NO: 17.

In some embodiments of the foregoing methods, the contact takes place in a host cell. In some embodiments, the host cell is chosen from the group consisting of: bacteria, yeast, or fungus. In some embodiments the host cell is a bacterium. In some embodiments, the bacterium is selected from the group consisting of: Escherichia, Bacillus, Corynebacteria, Methanococcus and Clostridium. In some embodiments, the host cell is yeast. In some embodiments, the yeast is selected from the group consisting of: Saccharomyces, Kluyveromyces, Candida, Yarrowia and Pichia. In some embodiments the host cell is a fungus. In some embodiments, the fungus is selected from the group consisting of: Aspergillus, Penicillium and Rhizobium.

In still another aspect, the disclosure provides methods of producing a 6-carbon keto acid selective decarboxylase. The methods include a) contacting a host cell with a nucleic acid encoding a polypeptide as described herein and; b) isolating the polypeptide from the host cell.

In some embodiments of the foregoing methods, the host cell is chosen from the group consisting of: bacteria, yeast, or fungus. In some embodiments the host cell is a bacterium. In some embodiments, the bacterium is selected from the group consisting of: Escherichia, Bacillus, Corynebacteria, Methanococcus and Clostridium. In some embodiments, the host cell is yeast. In some embodiments, the yeast is selected from the group consisting of: Saccharomyces, Kluyveromyces, Candida, Yarrowia and Pichia. In some embodiments, the host cell is a fungus. In some embodiments, the fungus is selected from the group consisting of: Aspergillus, Penicillium and Rhizobium.

### BRIEF DESCRIPTION OF THE DRAWINGS

FIG. 1 is schematic diagram of exemplary decarboxylase reactions.

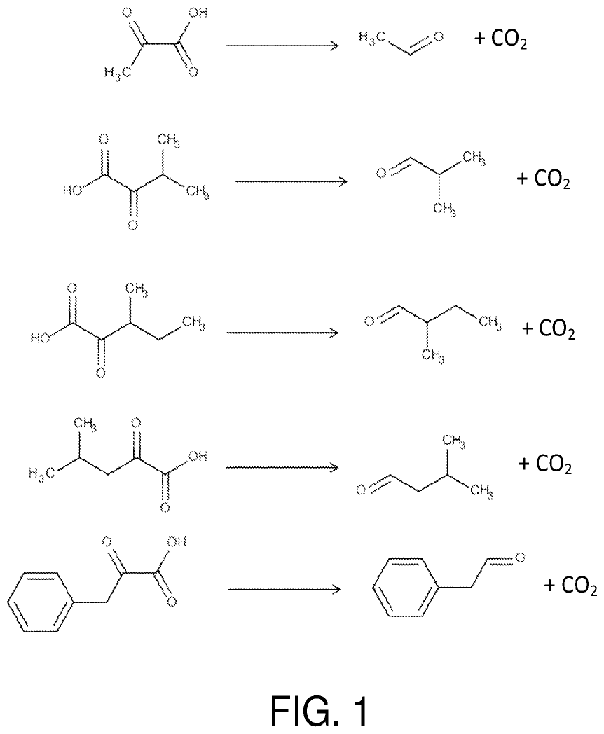

FIG. 2 is an illustration of isoleucine biosynthetic pathway in yeast.

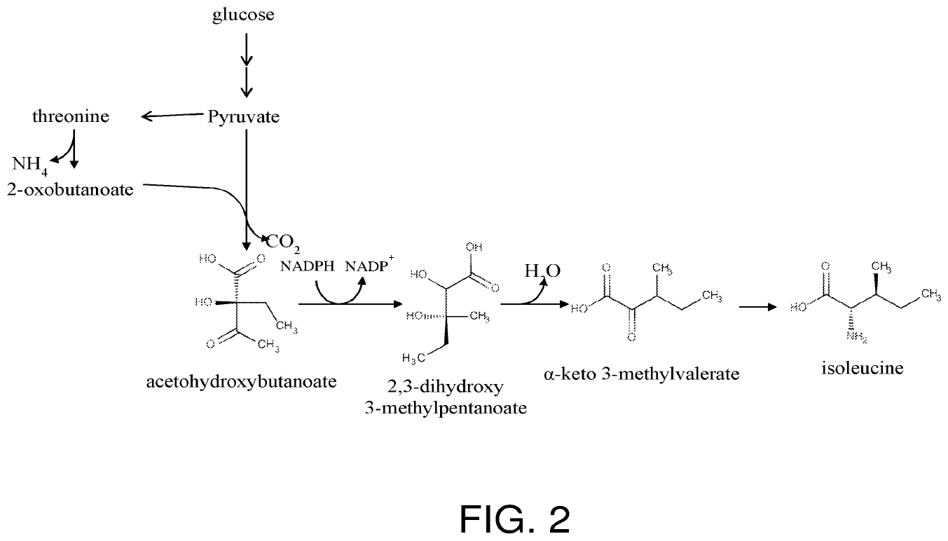

FIG. 3 is an illustration of leucine biosynthetic pathway in yeast.

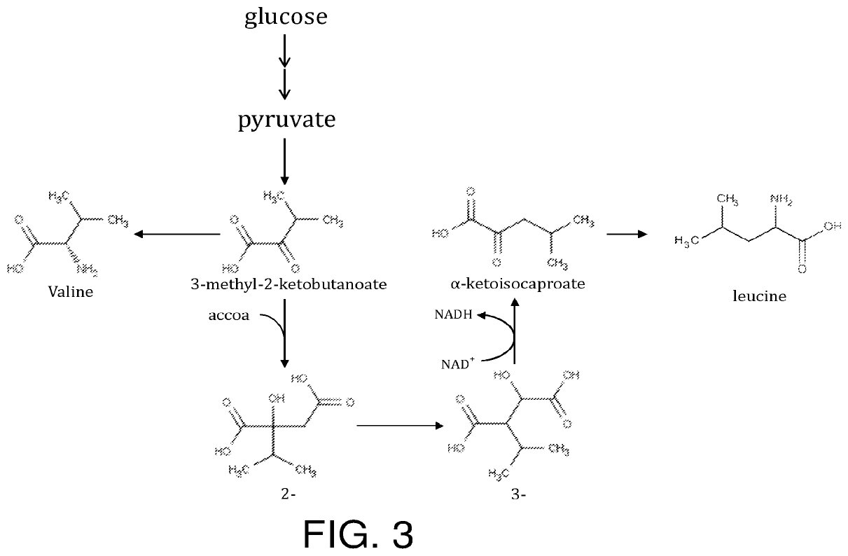

FIG. 4 is a diagram of the active site of decarboxylase corresponding to SEQ ID NO:

FIG. 5 is total ion chromatogram showing detection of isoamyl alcohols.

FIG. 6 is a bar graph showing the ratio of Vmax/Km of the different mutants corresponding to the parent enzyme having amino acid sequence of SEQ ID NO: 1 towards α-ketoisocaproic acid.

FIG. 7 is a bar graph showing the ratio of Vmax/Km of the different mutants corresponding to the parent enzyme having amino acid sequence of SEQ ID NO: 1 towards α-keto-3-methylvaleric acid.

FIG. 8 is a bar graph showing production of isoamyl alcohol (mg/L) using non-natural α-Ketoisocaproic acid decarboxylase and non-natural α-Keto-3-methylvaleric acid decarboxylase derived from SEQ ID NO: 3.

FIG. 9 is a bar graph showing production of isoamyl alcohol (mg/L) using non-natural α-Ketoisocaproic acid decarboxylase and non-natural α-Keto-3-methylvaleric acid decarboxylase derived from SEQ ID NO: 1.

### DETAILED DESCRIPTION

The present disclosure relates to engineered decarboxylases that have high specificity to α-ketoisocaproic acid or α-keto-3-methylvaleric acid. The disclosure also relates to non-natural microorganisms that host the engineered decarboxylases such that the non-natural microorganism can transcribe the gene to encode for the corresponding engineered decarboxylase. The present disclosure, therefore, provides means to design a non-natural decarboxylase that is highly specific to α-ketoisocaproic acid or α-keto-3-methylvaleric acid.

As used herein, the terms “polypeptide”, “peptide”, “protein” or “enzyme” are used interchangeably. In some embodiments, the catalytic promiscuity of some enzymes may be combined with protein engineering and may be exploited in novel metabolic pathways and biosynthesis applications. Protein engineering may result in a modification or improvement in the enzyme properties that may arise from the alteration in the structure-function of the enzyme and/or its interaction with other molecules. The interaction of an enzyme with other molecules such as for example the substrate can be quantified by the Michaelis constant (Km), which can be quantified using prior art (see for example, Stryer, Biochemistry, 4^{th} edition, W.H. Freeman, Nelson and Cox, Lenhinger Principles of Biochemistry, 6^{th} edition, W.H. Freeman) Conventional enzyme kinetics teaches that the product of the enzyme rate constant (kcat) and the concentration of the enzyme gives Vmax, which can be experimentally determined. The higher the kcat, the more substrate molecules get turned over in one second. The ratio of kcat/Km provides a quantitative measure of enzyme specificity and efficiency with a given substrate. Assuming the same amount of enzyme used across all measurements, the ratio of Vmax/Km will indicate the enzyme specificity for a specific substrate. Therefore, in accordance with this convention, enzyme specificity to a substrate can be quantified by the ratio of Vmax/Km for that substrate. The higher the value of this ratio, the more specific the enzyme is for that substrate.

As used herein, mutating or mutagenizing an amino acid is referred to the method of changing the amino acid in the parent sequence to another, different amino acid by altering the DNA sequence of the corresponding codon in the gene that is most likely to translate into the different amino acid.

The term “mutant” or “variant”, as used herein, refers to a protein or polypeptide in which one or more amino acid substitutions, deletions, and/or insertions are present as compared to the amino acid sequence of a protein or peptide, and includes naturally occurring allelic variants or alternative splice variants of an protein or peptide. The term “variant” includes the replacement of one or more amino acids in a peptide sequence with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987.) Preferred variants include alanine substitutions of one amino acid for another at one or more of amino acid positions. Other preferred substitutions include conservative substitutions that have little or no effect on the overall net charge, polarity, or hydrophobicity of the protein. Conservative substitutions are set forth in the table below.

| | |
|---|---|
| Basic: | arginine |
| | lysine |
| | histidine |
| Acidic: | glutamic acid |
| | aspartic acid |
| Uncharged Polar: | glutamine |
| | asparagine |
| | serine |
| | threonine |
| | tyrosine |
| Non-Polar: | phenylalanine |
| | tryptophan |
| | cysteine |
| | glycine |
| | alanine |
| | valine |
| | praline |
| | methionine |
| | leucine |
| | isoleucine |

The table below sets out another scheme of amino acid substitution:

| Original Residue | Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr; Ile |
| Phe | Met; Leu; Tyr |
| Ser | Thr |
| Thr | Ser |
| Trp | Tyr |
| Tyr | Trp; Phe |
| Val | Ile; Leu |

Other variants can consist of less conservative amino acid substitutions, such as selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to have a more significant effect on function are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine. Other variants include those designed to either generate a novel glycosylation and/or phosphorylation site(s), or those designed to delete an existing glycosylation and/or phosphorylation site(s). Variants include at least one amino acid substitution at a glycosylation site, a proteolytic cleavage site and/or a cysteine residue. Variants also include proteins and peptides with additional amino acid residues before or after the protein or peptide amino acid sequence on linker peptides. The term “variant” also encompasses polypeptides that have the amino acid sequence of the proteins/peptides of the present invention with at least one and up to 25 (e.g., 5, 10, 15, 20) or more (e.g., 30, 40, 50, 100) additional amino acids flanking either the 3′ or 5′ end of the amino acid sequence.

A keto acid, as used herein, is an organic compound containing a carboxylic acid group and a ketone group.

Those skilled in the art will understand that the herein disclosed decarboxylase designs are described in relation to, but are not limited to, species-specific genes and proteins and that the disclosure provides homologs and orthologs of such gene and protein sequences. The term “homology” of two sequences when used herein relates to the number of positions with identical nucleotides or amino acids divided by the number of nucleotides or amino acids in the shorter of the sequences, when the two sequences are aligned. Homolog and ortholog sequences possess a relatively high degree of sequence identity (i.e. from about 85% to about 100% sequence identity) when aligned using methods known in the art. Algorithms well known to those skilled in the art, such as Align, BLAST, Clustal W and others compare and determine a raw sequence similarity or identity. A computer comparison of two or more sequences can, if desired, also be optimized visually by those skilled in the art. Related gene products or proteins can be expected to have a high similarity, for example, 85% to 100% sequence identity. In some embodiments, useful polypeptide sequences have at least 85%, at least 90%, at least 91%, at least 92%, at least 93%, at least 94%, at least 95%, at least 96%, at least 97%, at least 98% or at least 99% identity to the amino acid sequence of the reference enzyme of interest.

The sequences, including those naturally occurring as well as engineered, disclosed here are intended to endow the microorganism with the ability to catalyze the desired reaction. It is understood that other enzymes that can catalyze the desired reactions are also within the scope of the disclosure. The skilled person will readily recognize that such enzymes may have a sequence identity of at least 25%, at least 30%, at least 35%, at least 40%, at least 45%, at least 50%, at least 55% or at least 60%, or at least 70%, or at least 80%, at least 90%, at least 91%, at least 92%, at least 93%, at least 94%, at least 95%, at least 96%, at least 97%, at least 98% or at least 99% to any given enzyme that is disclosed and will understand that they are not excluded from this disclosure.

As used herein, the acid and its conjugated base are used interchangeably, and refer to the molecule in context. For example, “α-Ketoisocaproic acid” and “α-ketoisocaproate” refer to the same chemical. As used herein, α-Ketoisocaproic acid may also be referred to as 2-keto-4-methyl-pentanoate, 2-oxoisocaproate, 2-oxo-4-methylpentanoate, α-ketoisocaproate, α-oxoisocaproate, 2-ketoisocaproate or keto-leucine. As used herein, α-keto-3-methylvaleric acid is also referred to as (3S)-3-methyl-2-oxopentanoate, α-keto-methylvalerate, 2-oxo-3-methylvalerate, (S)-2-oxo-3-methylpentanoate, (S)-3-methyl-2-oxovalerate, 2-oxo-3-methylpentanoate, 3-methyl-2-oxopentanoate, α-keto-β-methyl-valerate, 2-keto-3-methyl-valerate or keto-isoleucine.

As used herein, an engineered microorganism is one that is genetically modified from its corresponding wild-type. For example, the genetic modification could be one or more of: (i) introduction of exogenous nucleic acid sequences; (ii) introduction of additional copies of endogenous sequences; (iii) deletion of endogenous sequences and (iv) alteration of promoter or terminator sequences.

In some embodiments, wherein the microorganism has a cytoplasm, the microorganism may be further engineered to produce at least a portion, or at least a majority, or at least almost entirely, the target chemical in the cytoplasm. Identification and deletion of mitochondrial signal sequence to direct proteins into the cytosol is well-documented in the art (e.g. Strand M K, Stuart G R, Longley M J, Graziewicz M A, Dominick O C, Copeland W C (2003) POS5 gene of Saccharomyces cerevisiae encodes a mitochondrial NADH kinase required for stability of mitochondrial DNA. Eukaryot Cell 2:809-820; www.cbs.dtu.dk/services/; ihg.gsf.de/ihg/mitoprot.html).

Rational Engineering of Decarboxylases

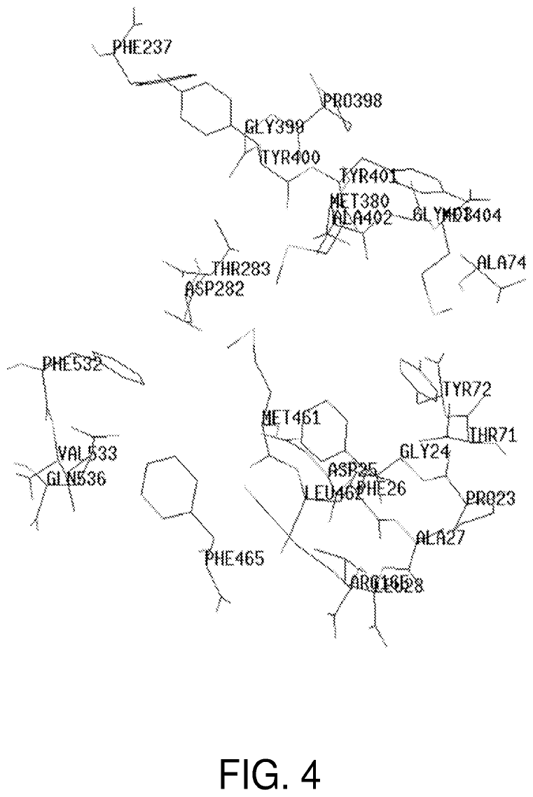

In some embodiments, the sequence of the parent α-ketoisocaproic acid decarboxylase or α-keto-3-methylvaleric acid decarboxylase is provided. In some embodiments, the non-natural protein sequence is created by enhancing the activity of α-ketoisocaproic acid decarboxylase or α-keto-3-methylvaleric acid decarboxylase by introducing one or more enzymes comprising an amino acid sequence having at least 85%, at least 90%, at least 95%, at least 96%, at least 97%, at least 98%, at least 99% identity to a sequence selected from SEQ ID NOS: 1-5. The crystal structure of the decarboxylase from Azospirillum brasilense identified several residues that have an impact on the substrate selectivity as well as the volume of the active site pocket Amino acids at the positions 23-28, 71, 72, 74, 112, 113, 165 from chain A, 237, 282, 283, 380, 385, 398-404, 461, 462, 465, 532, 533, 536 and 540 of SEQ ID NO: 3 are in close proximity to the active site of the enzyme and are implicated in determining the specificity and rate of the decarboxylase. These residues are shown in FIG. 4. In some embodiments, amino acids in at least one of the said positions are mutated into another amino acid.

In some embodiments, amino acids at the positions 286, 377, 381, 461, 465, 538 and 542 of SEQ ID NO: 1 are in close proximity to the active site of the enzyme and are implicated in determining the specificity and rate of the decarboxylase. In some embodiments, amino acids in at least one of these positions are mutated into another amino acid.

In some embodiments, at least one of the amino acids at the position corresponding to 110, 461, 377, 286, 538, 542 or 402 of SEQ ID NO: 1 are mutated to enhance the decarboxylation specificity to α-ketoisocaproic acid or α-keto-3-methylvaleric acid. In some embodiments, at least one of the amino acids at the position corresponding to 292, 288 or 476 of SEQ ID NO: 2 are mutated to enhance the decarboxylation specificity to α-ketoisocaproic acid or α-keto-3-methylvaleric acid. In some embodiments, at least one of the amino acids at the position corresponding to 380, 402 or 461 of SEQ ID NO: 3 are mutated to enhance the decarboxylation specificity to α-ketoisocaproic acid or α-keto-3-methylvaleric acid. In some embodiments, at least one of the amino acids at the position corresponding to 290, 388, 392 or 472 of SEQ ID NO: 4 are mutated to enhance the decarboxylation specificity to α-ketoisocaproic acid or α-keto-3-methylvaleric acid. In some embodiments, at least one of the amino acids at the position corresponding to 444, 469 or 544 of SEQ ID NO: 5 are mutated to enhance the decarboxylation specificity to α-ketoisocaproic acid or α-keto-3-methylvaleric acid. Whether a polypeptide has the desired decarboxylase activity or not may be determined by in vitro assays as illustrated in the examples.

In some embodiments, the modified decarboxylases are further engineered by subjecting them to random mutagenesis. The modified and mutagenized decarboxylases are selectively identified by selecting for higher specificity to α-ketoisocaproic acid or α-keto-3-methylvaleric acid.

In some embodiments, the engineered decarboxylases are expressed in conjunction with an alcohol dehydrogenase or oxidoreductase to convert the product of the decarboxylation reaction into the corresponding alcohol. In some embodiments, the alcohols derived from α-ketoisocaproic acid and α-keto-3-methylvaleric acid are 3-methylbutanol and 2-methylbutanol, respectively. A “dehydrogenase” is an enzyme that catalyzes the removal of hydrogen atoms from a molecule by a reduction reaction that removes one or more hydrogens from a substrate to an electron acceptor, such as NAMNADP⁺ or a flavin coenzyme, such as FAD or FMN. An “oxidoreductase” is an enzyme that catalyzes the transfer of electrons from one molecule, the redundant, also called the electron donor, to another, the oxidant, also called the electron acceptor.

In some embodiments, the engineered decarboxylases are expressed in conjunction with an aldehyde dehydrogenase to convert the product of the decarboxylation reaction into the corresponding carboxylic acid. In some embodiments, the carboxylic acid derived from α-ketoisocaproic acid and α-keto-3-methylvaleric acid are 3-methylbutyric acid and 2-methylbutyric acid, respectively.

In some embodiments, the engineered decarboxylase and the dehydrogenase enzymes are expressed from a suitable host cell. The host cell is selected from a eukaryotic cell, bacteria or archaea. Examples of eukaryotic cells include, but are not limited to, Pichia (such as Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia kudriavzevii), Saccharomyces (such as Saccharomyces cerevisiae), Hansenula polymorpha, Kluyveromyces (such as Kluyveromyces lactis, Kluyveromyces marxianus), Candida albicans, Aspergillus (such as Aspergillus nidulans, Aspergillus niger, Aspergillus oryzae), Trichoderma reesei, Chrysosporium lucknowense, Fusarium (such as Fusarium gramineum, Fusarium venenatum), Neurospora crassa, Yarrowia lipolytica, and Chlamydomonas reinhardtii, and the like. Examples of bacteria include, but are not limited to, Acinetobacter (such as Acinetobacter baylyi), Agrobacterium, Alicyclobacillus, Anabaena, Anacystis, Arthrobacter, Azobacter, Bacillus (such as Bacillus subtilis, Bacillus amyloliquefacines), Brevibacterium (such as Brevibacterium ammoniagenes, Brevibacterium immariophilum), Chromatium, Clostridium (such as Clostridium beijerinckii), Corynebacterium, Enterobacter (such as Enterobacter sakazakii), Erwinia, Escherichia (such as Escherichia coli), Lactobacillus, Lactococcus (such as Lactococcus lactis), Mesorhizobium (such as Mesorhizobium loti), Methylobacterium, Microbacterium, Phormidium, Pseudomonas (such as Pseudomonas aeruginosa, Pseudomonas citronellolis, Pseudomonas mevalonii, Pseudomonas pudica), Rhodobacter (such as Rhodobacter capsulatus, Rhodobacter sphaeroides), Rhodopseudomonas, Rhodospirillum (such as Rhodospirillum rubrum), Rhodococcus, Salmonella (such as Salmonella enterica, Salmonella typhi, Salmonella typhimurium), Scenedesmun, Serratia, Shigella (such as Shigella dysenteriae, Shigella flexneri, Shigella sonnei), Staphylococcus (such as Staphylococcus aureus), Streptomyces, Synnecoccus, Zymomonas, and the like. Examples of archaea include, but are not limited to Aeropyrum (such as Aeropyrum pemix), Archaeglobus (such as Archaeoglobus fulgidus), Halobacterium, Methanococcus (such as Methanococcus jannaschii), Methanobacterium (such as Methanobacterium thermoautotrophicum), Pyrococcus (such as Pyrococcus abyssi, Pyrococcus horikoshii), Sulfolobus, and Thermoplasma (such as Thermoplasma acidophilum, Thermoplasma volcanium), and the like.

### EXAMPLES

The following examples are provided only as a means to further illustrate the compositions and methods described herein.

### Example 1

Enzyme assays were performed with the decarboxylase mutants to test for enhanced specificity to α-ketoisocaproic acid and α-keto-3-methylvaleric acid. The nucleotide sequences corresponding to SEQ ID NOs: 6-12 were derived from the nucleotide sequence encoding for a polypeptide that has the amino acid sequence of SEQ ID NO: 1 using Q5 Site-Directed Mutagenesis Kit (New England Biolabs, Ipswich, Mass.). Successful incorporation of the mutation at the desired location was confirmed by Sanger sequencing. The BW25113 strain of Escherichia coli was used for all enzyme assays. Decarboxylase genes were expressed from the constitutive lac promoter from a plasmid vector. The E. coli cells containing the decarboxylase mutants were grown in 50 mL LB medium until the mid-log phase, in 250 mL shake flasks (37° C., 200 rpm) and harvested by centrifugation and frozen at −80° C. Subsequently, the soluble proteins were extracted with B-PER™ Bacterial Protein Extraction Reagent (Thermo Fisher Scientific), following the manufacturer's protocol. The cell lysate obtained was used for the in vitro coupled enzymatic assay, which was performed by reducing the product of the decarboxylation reaction to the corresponding alcohol by monitoring the depletion of NADH at 340 nm. The assay was performed in 50 mM phosphate buffer (pH 6.5) supplemented with 1 mM MgCl₂. Reaction was set up with the following ingredients: 2.5 U/mL of the equine alcohol dehydrogenase (Sigma-Aldrich), 0.35 mg/mL of NADH, 0.23 mg/mL of thiamine pyrophosphate (ThPP) and a substrate (α-ketoisocaproic acid, α-keto-3-methylvaleric acid, α-ketoisovaleric acid and pyruvate) ranging in concentration from 0.05 to 0.3 g/L. The assay was performed in 96-well plates using the Spectra Max Plus 384 plate reader (Molecular Devices). The slope of the time vs absorbance curve during the steady state stage of the reaction was determined from the raw data. Km and Vmax were determined using the Lineweaver-Burk double reciprocal plot (1/S vs 1/V) and the Vmax/Km ratio was calculated.

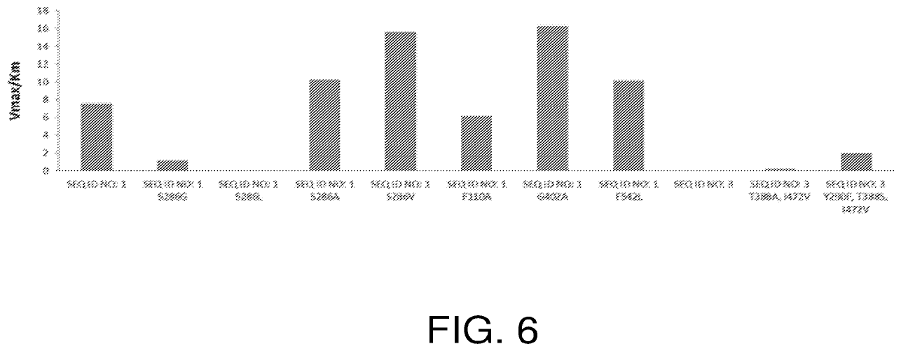

The parent enzyme from which the engineered decarboxylases were derived was classified as α-ketoisovaleric acid decarboxylase and had a Vmax/Km value of 0.67 for its native substrate. The value of Vmax/Km for the various engineered decarboxylase mutants using α-ketoisocaproic acid are shown in FIG. 6.

As an example of enhancing the specificity of the decarboxylase to α-ketoisocaproic acid, the polypeptide with SEQ ID NO: 1 was engineered by mutating amino acids at position S286A, S286V and G402A. These single amino acid mutants had significantly higher Vmax/Km ratio for α-ketoisocaproic acid compared to the parent decarboxylase as well as other mutants. The value of Vmax/Km for the various engineered decarboxylase mutants using α-ketoisocaproic acid are shown in FIG. 6.

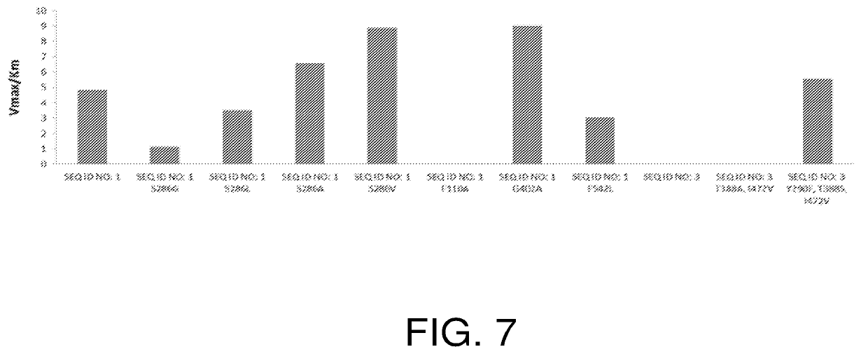

As an example of enhancing the specificity of the decarboxylase to α-keto-3-methylvaleric acid, the polypeptide of SEQ ID NO: 1 was engineered by mutating amino acids at position S286V and G402A. Similarly, the polypeptide with SEQ ID NO: 3 was engineered to contain the Y290F, T388S, I472V mutations. These engineered mutants exhibited significantly higher Vmax/Km ratio compared to the parent as well as other mutants for α-keto-3-methylvaleric acid. The value of Vmax/Km for the various engineered decarboxylase mutants using α-keto-3-methylvaleric acid are shown in FIG. 7.

### Example 2

The yeast Kluyveromyces lactis strain GG799 (New England Biolabs, Ipswich, Mass.) was used as the host organism. Codon-optimized DNA sequences corresponding to the desired amino acid sequences were de novo synthesized by GenScript (Piscataway, N.J.) and were sub-cloned into HindIII/XhoI sites of pKlac2 shuttle vector from New England Biolabs (Ipswich, Mass.). The synthetic genes were integrated at the LAC4 locus using K. lactis Protein Expression Kit New England Biolabs (Ipswich, Mass.). The genetic modification was verified by colony PCR. The native keto-acid decarboxylase gene was amplified by using ATGTACACTGTTGGTGATTACTTG (SEQ ID NO: 23) and TTAAGACTTGTTTTGTTCAGCGAAC (SEQ ID NO: 24) as the forward and reverse primers, respectively. The recombinant microorganisms, thus created and verified, were stored at −80° C. as glycerol stocks using YPD broth.

The recombinant microorganisms were prepared from the stocks. The cultures were incubated in culture tubes containing 3 mL of broth in a shaker-incubator at 30° C. at 250 rpm overnight. These initial cultures were used to inoculate 250 mL shake flasks containing 50 mL of freshly-prepared minimal medium containing 30 g/L glucose. The flasks were incubated in a shaker at 30° C. at 250 rpm for 15 hrs. Final optical densities (OD600) of these seed cultures were in 8.2-9.5 range. The cells were centrifuged at 7,000 g for 5 min and resuspended in 6 mL of freshly-prepared CBS medium containing 30 g/L galactose. This concentrated cell slurry was used to inoculate 250 mL shake flasks with 30 g/L galactose such that the initial optical density (600 nm) of the galactose cultures was 3. The flasks were placed in a shaker-incubator at 30° C. at 100 rpm for 3 hrs. At the end of 3 h, 2 mL samples of all of the cultures were withdrawn and optical density (600 nm) of the samples was measured. The samples were centrifuged at 14,000 g for 5 min to separate the cells from the supernatant. The supernatants were stored frozen at −80° C. for further analysis.

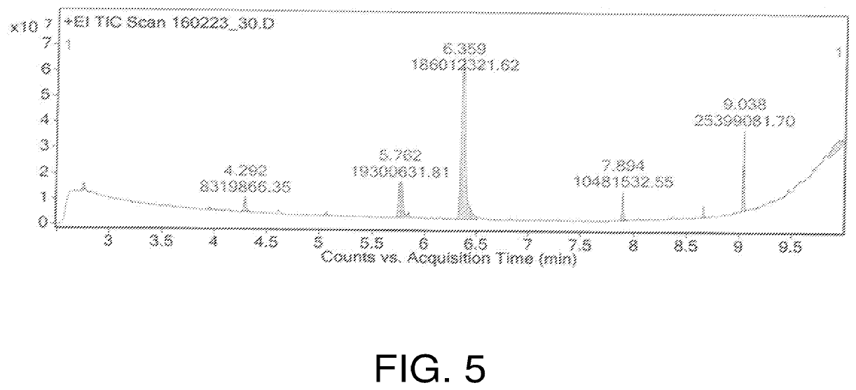

The supernatant was thawed and n-pentanol was added as an internal standard for the analysis and vortexed with equal volume of diethyl ether to extract n-pentanol, 2-methylbutanol and 3-methylbutanol. The organic phase was removed and injected into GC for analysis. FIG. 5 indicates the detection of 2-methylbutanol and 3-methylbutanol at a retention time of 5.2 min.

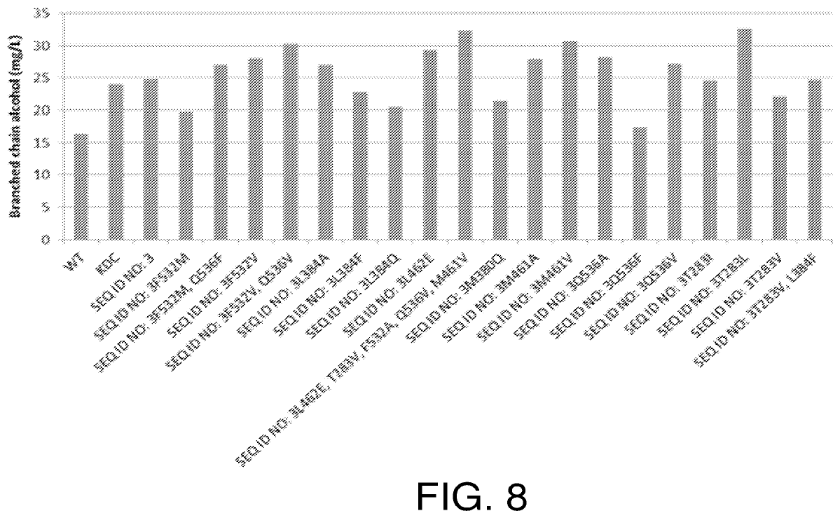

A polynucleotide sequence having the sequence shown of SEQ ID NO: 3 was synthesized de novo. Mutations were introduced in the corresponding nucleotide sequence using site-directed mutagenesis. These mutants were introduced in K. lactis and the supernatant was analyzed for the presence of 2-methylbutanol and 3-methylbutanol. As indicated in FIG. 8, the total amount of 2-methylbutanol and 3-methylbutanol produced by the engineered decarboxylases is more than that produced by the non-engineered control. The WT column in FIG. 8 shows total concentration of the two alcohols for yeast that had no gene introduced. The KDC column in FIG. 8 shows total concentration of the two alcohols for yeast with overexpression of native KDC gene. The SEQ ID NO: 3 column in FIG. 8 shows total concentration of the two alcohols for yeast transformed with a gene that encodes a protein with the amino acid sequence of SEQ ID NO: 3. The remaining columns show total concentration of the two alcohols for yeast transformed with a gene that encodes a protein with the amino acid sequence of SEQ ID NO: 3 containing the listed amino acid mutations.

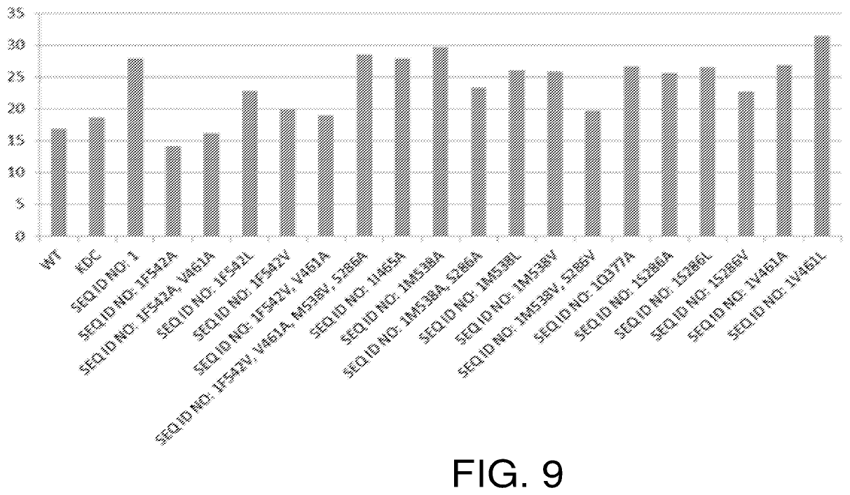

As indicated in FIG. 9, the total amount of 2-methylbutanol and 3-methylbutanol produced by some decarboxylases engineered from SEQ ID NO: 1 is more than that produced by the non-engineered control. The WT column in FIG. 9 shows total concentration of the two alcohols for yeast that had no gene introduced. The KDC column in FIG. 9 shows total concentration of the two alcohols for yeast transformed with the native KDC gene. The SEQ ID NO: 1 column in FIG. 9 shows total concentration of the two alcohols for yeast transformed with a gene that encodes a protein with the amino acid sequence of SEQ ID NO: 1. The remaining columns show total concentration of the two alcohols for yeast transduce with a gene that encodes a protein with the amino acid sequence of SEQ ID NO: 1 with the listed amino acid mutations.

1.-44. (canceled)

45. A polypeptide exhibiting 6-carbon keto acid specific decarboxylase activity comprising a variant chosen from a first variant and a second variant, wherein the first variant is a variant of the amino acid sequence of SEQ ID NO: 3, wherein the first variant comprises 1-40 amino acid substitutions or deletions when compared to the amino acid sequence of SEQ ID NO: 3, and the second variant is a variant of the amino acid sequence of SEQ ID NO: 1, wherein the second variant comprises 1-40 amino acid substitutions or deletions when compared to the amino acid sequence of SEQ ID NO: 1.

46. A polypeptide according to claim 45, wherein the polypeptide has a level of activity associated with the 6-carbon keto-acid decarboxylase activity that is greater than a level of activity associated with a polypepride having 5-carbon keto-acid decarboxylase activity, 4-carbon keto-acid decarboxylase activity, or 3-carbon keto-acid decarboxylase activity.

47. A polypeptide according to claim 45, wherein the polypeptide has a level of activity associated with the 6-carbon keto-acid decarboxylase activity that is greater than a level of activity associated with a poypeptide having 7-carbon keto-acid decarboxylase activity or an 8-carbon keto-acid decarboxylase activity.

48. A polypeptide according to claim 45, wherein the 6-carbon keto acid is α-ketoisocaproic acid decarboxylase.

49. A polypeptide according to claim 45, wherein the 6-carbon keto acid is α-keto-3-methylvaleric acid decarboxylase.

50. A polypeptide according to claim 45, wherein:
a) the substitutions of the first variant occur at a position of SEQ ID NO: 3 chosen from: L462, T283, L384, M380, Q536, M461 and F532; and,
b) the substitutions of the second variant occur at a position of SEQ ID NO: 1 chosen from: F110, F542, Q377, V461, M538, 5286, F381 and 1465.

51. A polypeptide according to claim 50, wherein:
a) the first variant amino acid substitution is chosen from: Glu, Ile, Val, Ala, Gln, Met, Leu, Ser, and Phe; and,
b) the second variant amino acid substitution is chosen from Ala, Leu, Gly, and Val.

52. A polypeptide according to claim 45, wherein:
a) the first variant amino acid substitution is at least one substitution chosen from: F532A, F532M, F532M/Q536F, F532V, F532V/Q536V, L384A, L384F, L384Q, L462E, L462E/T283V/F532A/Q536V/M461V, M380Q, M461A, M461V, Q536A, Q536F, Q536V, T283I, T283L, T283V, T388A/I472V, Y290F/T388S/1472V, and T283V/L384F; and,
b) the second variant amino acid substitution is at least one substitution chosen from: F110A, F542A, F542A/V461A, F542L, F542V, F542V/V461A, F542V/V461A/M538V/S286A, I465A, M538A, M538A/S286A, M538L, M538V, M538V/S286V, Q377A, S286A, S286L, S286V, V461A, S286G, F110A, G402A, F542L and V461L.

53. A polypeptide according to claim 52, wherein:
a) the first variant amino acid substitution is at least one substitution chosen from: F532M/Q536F, F532V, F532V/Q536V, L384A, L462E, L462E/T283V/F532A/Q536V/M461V, M461A, M461V, Q536A, Q536V, and T283L; and,
b) the second variant amino acid substitution is at least one substitution chosen from: F542V/V461A/M538V/S286A, I465A, F110A, M538A and V461L

54. A polypeptide according to claim 45, wherein the first variant comprises 1-7 amino acid substitutions or deletions when compared to the amino acid sequence of SEQ ID NO: 3 and the second variant comprises 1-7 amino acid substitutions or deletions when compared to the amino acid sequence of SEQ ID NO:1.

55. A polypeptide according to claim 45, wherein the first variant comprises at least 94% homology with the amino acid sequence of SEQ ID NO: 3, and the second variant comprising at least 94% homology with the amino acid sequence of SEQ ID NO: 1.

56. A method of producing an aldehyde comprising contacting a 6-carbon keto acid with the polypeptide of claim 45.

57. A method according to claim 12, wherein the 6-carbon keto acid is chosen from: α-ketoisocaproic acid and α-keto-3-methylvaleric acid.

58. A method according to claim 12, further comprising producing a product chosen from: 2-methylbutanol, 3-methylbutanol, 2-methylbutyric acid, and 3-methylbutyric acid, wherein: producing the product comprises contacting the aldehyde with a dehydrogenase or oxidoreductase.

59. A method according to claim 14, wherein the product is 2-methylbutanol or 3-methylbutanol, the dehydrogenase is an alcohol dehydrogenase with at least 75% homology to SEQ ID NOS: 15 or 16 and when the product is 2-methylbutyric acid or 3-methylbutyric acid, the dehydrogenase is an aldehyde dehydrogenase with at least 75% homology to SEQ ID NO: 17.

60. A method according to claim 12, wherein contacting takes place in a host cell.

61. A method according to claim 16, wherein the host cell is chosen from: bacteria, yeast, and fungus, and further wherein the bacteria is chosen from: Escherichia, Bacillus, Corynebacteria, Methanococcus and Clostridium, the yeast is chosen from: Saccharomyces, Kluyveromyces, Candida, Yarrowia and Pichia, and the fungus is chosen from: Aspergillus, Penicillium and Rhizobium.

62. A method of producing a 6-carbon keto acid selective decarboxylase comprising contacting a host cell with a nucleic acid encoding a polypeptide according to claim 45.

63. A method according to claim 62, further comprising isolating the polypeptide from the host cell.

64. A method according to claim 62, wherein the host cell is chosen from: bacteria, yeast, and fungus, and further wherein the bacteria is chosen from: Escherichia, Bacillus, Corynebacteria, Methanococcus and Clostridium, the yeast is chosen from: Saccharomyces, Kluyveromyces, Candida, Yarrowia and Pichia, and the fungus is chosen from: Aspergillus, Penicillium and Rhizobium.


SEQUENCE LISTING (24 sequences)

SEQ ID NO: 1 - protein, 548 aa, Lactococcus lactis
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENPTSNTSDQEILNKIQESLKNAKKPIVITGHEIISFGLENTVTQFISKTKLPITTLNFGKSSVDETLPSFLGIYNGKLSEPNLKEFVESADFILMLGVKLTDSSTGAFTHHLNENKMISLNIDEGKIFNESIQNFDFESLISSLLDLSGIEYKGKYIDKKQEDFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELVLAKEDAPKVLKKMGKLFAEQNKS

SEQ ID NO: 2 - protein, 563 aa, Kluyveromyces lactis
MSEITLGRYLFERLKQVEVQTIFGLPGDFNLSLLDNIYEVPGMRWAGNANELNAAYAADGYARLKGMSCIITTFGVGELSALNGIAGSYAEHVGVLHVVGVPSVSSQAKQLLLHHTLGNGDFTVFHRMSSNISETTAMITDINTAPAEIDRCIRTTYVSQRPVYLGLPANLVDLTVPASLLDTPIDLSLKPNDPEAEEEVIENVLQLIKEAKNPVILADACCSRHDAKAETKKLIDLTQFPAFVTPMGKGSIDEKHPRFGGVYVGTLSSPAVKEAVESADLVLSVGALLSDFNTGSFSYSYKTKNIVEFHSDYTKIRSATFPGVQMKFALQKLLTKVADAAKGYKPVPVPSEPEHNEAVADSTPLKQEWVWTQVGEFLREGDVVITETGTSAFGINQTHFPNNTYGISQVLWGSIGFTTGATLGAAFAAEEIDPKKRVILFIGDGSLQLTVQEISTMIRWGLKPYLFVLNNDGYTIERLIHGETAQYNCIQNWQHLELLPTFGAKDYEAVRVSTTGEWNKLTTDEKFQDNTRIRLIEVMLPTMDAPSNLVKQAQLTAATNAKN

SEQ ID NO: 3 - protein, 545 aa, Azospirillum brasilense
MKLAEALLRALKDRGAQAMFGIPGDFALPFFKVAEETQILPLHTLSHEPAVGFAADAAARYSSTLGVAAVTYGAGAFNMVNAVAGAYAEKSPVVVISGAPGTTEGNAGLLLHHQGRTLDTQFQVFKEITVAQARLDDPAKAPAEIARVLGAARALSRPVYLEIPRNMVNAEVEPVGDDPAWPVDRDALAACADEVLAAMRSATSPVLMVCVEVRRYGLEAKVAELAQRLGVPVVTTFMGRGLLADAPTPPLGTYIGVAGDAEITRLVEESDGLFLLGAILSDTNFAVSQRKIDLRKTIHAFDRAVTLGYHTYADIPLAGLVDALLEGLPPSDRTTRGKEPHAYPTGLQADGEPIAPMDIARAVNDRVRAGQEPLLIAADMGDCLFTAMDMIDAGLMAPGYYAGMGFGVPAGIGAQCVSGGKRILTVVGDGAFQMTGWELGNCRRLGIDPIVILFNNASWEMLRTFQPESAFNDLDDWRFADMAAGMGGDGVRVRTRAELKAALDKAFATRGRFQLIEAMIPRGVLSDTLARFVQGQKRLHAAPRE

SEQ ID NO: 4 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDFSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAESGDSWFNAQRMKLPNGARVEYEMQWGHIGWSVPAAFGYAVGAPERRNILMVGDGSFQLTAQEVAQMVRLKLPVIIFLINNYGYTVEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 5 - protein, 635 aa, Saccharomyces cerevisiae
MAPVTIEKFVNQEERHLVSNRSATIPFGEYIFKRLLSIDTKSVFGVPGDFNLSLLEYLYSPSVESAGLRWVGTCNELNAAYAADGYSRYSNKIGCLITTYGVGELSALNGIAGSFAENVKVLHIVGVAKSIDSRSSNFSDRNLHHLVPQLHDSNFKGPNHKVYHDMVKDRVACSVAYLEDIETACDQVDNVIRDIYKYSKPGYIFVPADFADMSVTCDNLVNVPRISQQDCIVYPSENQLSDIINKITSWIYSSKTPAILGDVLTDRYGVSNFLNKLICKTGIWNFSTVMGKSVIDESNPTYMGQYNGKEGLKQVYEHFELCDLVLHFGVDINEINNGHYTFTYKPNAKIIQFHPNYIRLVDTRQGNEQMFKGINFAPILKELYKRIDVSKLSLQYDSNVTQYTNETMRLEDPTNGQSSIITQVHLQKTMPKFLNPGDVVVCETGSFQFSVRDFAFPSQLKYISQGFFLSIGMALPAALGVGIAMQDHSNAHINGGNVKEDYKPRLILFEGDGAAQMTIQELSTILKCNIPLEVIIWNNNGYTIERAIMGPTRSYNDVMSWKWTKLFEAFGDFDGKYTNSTLIQCPSKLALKLEELKNSNKRSGIELLEVKLGELDFPEQLKCMVEAAALKRNKK

SEQ ID NO: 6 - protein, 1647 aa, Artificial Sequence
  KDC2 mutant S286G
ATGTATACGGTGGGCGACTACCTGCTGGACCGTCTGCATGAGTTGGGAATTGAGGAAATCTTCGGCGTTCCCGGGGACTACAACTTGCAGTTTCTGGATCAAATCATTAGCCGCAAAGATATGAAATGGGTCGGCAATGCTAACGAGCTTAATGCAAGTTACATGGCCGACGGATATGCGCGTACTAAAAAAGCCGCAGCTTTCTTAACGACTTTCGGTGTGGGTGAACTTTCTGCAGTAAATGGGTTAGCTGGTTCATACGCCGAAAACTTACCAGTGGTGGAAATCGTCGGGTCGCCTACCTCAAAGGTCCAGAATGAAGGAAAATTTGTTCATCACACGCTGGCGGATGGAGACTTTAAGCATTTTATGAAAATGCACGAACCCGTTACAGCTGCCCGCACATTGCTGACAGCTGAAAACGCGACAGTGGAGATTGACCGTGTACTGAGTGCGCTGCTGAAGGAGCGCAAGCCTGTGTATATCAATCTGCCTGTCGACGTAGCAGCAGCGAAAGCTGAAAAGCCCTCATTACCACTGAAAAAGGAGAATCCCACTTCTAACACTAGCGACCAGGAAATCCTGAACAAAATCCAAGAATCGCTTAAGAACGCTAAAAAACCCATTGTCATTACAGGGCATGAGATTATTAGCTTTGGGCTTGAGAACACTGTAACTCAGTTTATTAGTAAGACGAAGTTACCCATCACAACCTTGAATTTTGGGAAGTCCTCCGTAGATGAAACCTTGCCCTCGTTCCTTGGTATCTACAACGGTAAACTGTCAGAGCCAAACCTTAAAGAGTTTGTGGAGTCGGCCGACTTCATTTTGATGTTGGGCGTGAAGCTGACTGACGGCTCTACAGGCGCGTTCACTCACCATTTAAATGAAAACAAGATGATCTCCCTTAACATCGACGAGGGCAAAATTTTTAATGAGTCGATTCAGAACTTCGACTTCGAGTCGTTAATTTCGTCCTTACTGGATCTTTCTGGTATTGAGTACAAAGGCAAGTACATTGACAAGAAGCAAGAAGATTTCGTCCCTAGCAACGCCTTACTTAGCCAGGACCGTCTTTGGCAGGCAGTAGAAAACTTGACGCAGTCTAATGAAACGATTGTTGCCGAACAGGGTACCTCCTTTTTCGGGGCGAGTAGTATTTTTTTGAAACCAAAGAGCCACTTTATCGGGCAACCATTATGGGGGTCCATTGGGTATACCTTCCCAGCTGCTTTAGGTTCCCAAATTGCAGACAAGGAGAGTCGTCATTTGCTGTTCATTGGTGATGGTTCATTGCAACTTACAGTGCAGGAACTGGGTTTAGCGATCCGTGAAAAAATTAACCCCATCTGCTTTATTATCAATAACGATGGTTATACCGTAGAGCGTGAAATTCATGGACCTAATCAATCGTATAATGATATTCCAATGTGGAACTACAGTAAGTTGCCTGAATCATTCGGCGCGACAGAGGAGCGCGTGGTCTCCAAAATCGTGCGCACAGAGAACGAATTCGTATCAGTGATGAAAGAAGCACAGGCCGACCCCAATCGCATGTATTGGATTGAACTGGTATTGGCGAAGGAAGACGCGCCTAAGGTACTGAAGAAGATGGGAAAACTTTTTGCTGAGCAAAACAAATCATAA

SEQ ID NO: 7 - protein, 1647 aa, Artificial Sequence
  KDC2 - mutant S286L
ATGTATACGGTGGGCGACTACCTGCTGGACCGTCTGCATGAGTTGGGAATTGAGGAAATCTTCGGCGTTCCCGGGGACTACAACTTGCAGTTTCTGGATCAAATCATTAGCCGCAAAGATATGAAATGGGTCGGCAATGCTAACGAGCTTAATGCAAGTTACATGGCCGACGGATATGCGCGTACTAAAAAAGCCGCAGCTTTCTTAACGACTTTCGGTGTGGGTGAACTTTCTGCAGTAAATGGGTTAGCTGGTTCATACGCCGAAAACTTACCAGTGGTGGAAATCGTCGGGTCGCCTACCTCAAAGGTCCAGAATGAAGGAAAATTTGTTCATCACACGCTGGCGGATGGAGACTTTAAGCATTTTATGAAAATGCACGAACCCGTTACAGCTGCCCGCACATTGCTGACAGCTGAAAACGCGACAGTGGAGATTGACCGTGTACTGAGTGCGCTGCTGAAGGAGCGCAAGCCTGTGTATATCAATCTGCCTGTCGACGTAGCAGCAGCGAAAGCTGAAAAGCCCTCATTACCACTGAAAAAGGAGAATCCCACTTCTAACACTAGCGACCAGGAAATCCTGAACAAAATCCAAGAATCGCTTAAGAACGCTAAAAAACCCATTGTCATTACAGGGCATGAGATTATTAGCTTTGGGCTTGAGAACACTGTAACTCAGTTTATTAGTAAGACGAAGTTACCCATCACAACCTTGAATTTTGGGAAGTCCTCCGTAGATGAAACCTTGCCCTCGTTCCTTGGTATCTACAACGGTAAACTGTCAGAGCCAAACCTTAAAGAGTTTGTGGAGTCGGCCGACTTCATTTTGATGTTGGGCGTGAAGCTGACTGACCTGTCTACAGGCGCGTTCACTCACCATTTAAATGAAAACAAGATGATCTCCCTTAACATCGACGAGGGCAAAATTTTTAATGAGTCGATTCAGAACTTCGACTTCGAGTCGTTAATTTCGTCCTTACTGGATCTTTCTGGTATTGAGTACAAAGGCAAGTACATTGACAAGAAGCAAGAAGATTTCGTCCCTAGCAACGCCTTACTTAGCCAGGACCGTCTTTGGCAGGCAGTAGAAAACTTGACGCAGTCTAATGAAACGATTGTTGCCGAACAGGGTACCTCCTTTTTCGGGGCGAGTAGTATTTTTTTGAAACCAAAGAGCCACTTTATCGGGCAACCATTATGGGGGTCCATTGGGTATACCTTCCCAGCTGCTTTAGGTTCCCAAATTGCAGACAAGGAGAGTCGTCATTTGCTGTTCATTGGTGATGGTTCATTGCAACTTACAGTGCAGGAACTGGGTTTAGCGATCCGTGAAAAAATTAACCCCATCTGCTTTATTATCAATAACGATGGTTATACCGTAGAGCGTGAAATTCATGGACCTAATCAATCGTATAATGATATTCCAATGTGGAACTACAGTAAGTTGCCTGAATCATTCGGCGCGACAGAGGAGCGCGTGGTCTCCAAAATCGTGCGCACAGAGAACGAATTCGTATCAGTGATGAAAGAAGCACAGGCCGACCCCAATCGCATGTATTGGATTGAACTGGTATTGGCGAAGGAAGACGCGCCTAAGGTACTGAAGAAGATGGGAAAACTTTTTGCTGAGCAAAACAAATCATAA

SEQ ID NO: 8 - protein, 1647 aa, Artificial Sequence
  KDC2 - mutant S286A
ATGTATACGGTGGGCGACTACCTGCTGGACCGTCTGCATGAGTTGGGAATTGAGGAAATCTTCGGCGTTCCCGGGGACTACAACTTGCAGTTTCTGGATCAAATCATTAGCCGCAAAGATATGAAATGGGTCGGCAATGCTAACGAGCTTAATGCAAGTTACATGGCCGACGGATATGCGCGTACTAAAAAAGCCGCAGCTTTCTTAACGACTTTCGGTGTGGGTGAACTTTCTGCAGTAAATGGGTTAGCTGGTTCATACGCCGAAAACTTACCAGTGGTGGAAATCGTCGGGTCGCCTACCTCAAAGGTCCAGAATGAAGGAAAATTTGTTCATCACACGCTGGCGGATGGAGACTTTAAGCATTTTATGAAAATGCACGAACCCGTTACAGCTGCCCGCACATTGCTGACAGCTGAAAACGCGACAGTGGAGATTGACCGTGTACTGAGTGCGCTGCTGAAGGAGCGCAAGCCTGTGTATATCAATCTGCCTGTCGACGTAGCAGCAGCGAAAGCTGAAAAGCCCTCATTACCACTGAAAAAGGAGAATCCCACTTCTAACACTAGCGACCAGGAAATCCTGAACAAAATCCAAGAATCGCTTAAGAACGCTAAAAAACCCATTGTCATTACAGGGCATGAGATTATTAGCTTTGGGCTTGAGAACACTGTAACTCAGTTTATTAGTAAGACGAAGTTACCCATCACAACCTTGAATTTTGGGAAGTCCTCCGTAGATGAAACCTTGCCCTCGTTCCTTGGTATCTACAACGGTAAACTGTCAGAGCCAAACCTTAAAGAGTTTGTGGAGTCGGCCGACTTCATTTTGATGTTGGGCGTGAAGCTGACTGACGCGTCTACAGGCGCGTTCACTCACCATTTAAATGAAAACAAGATGATCTCCCTTAACATCGACGAGGGCAAAATTTTTAATGAGTCGATTCAGAACTTCGACTTCGAGTCGTTAATTTCGTCCTTACTGGATCTTTCTGGTATTGAGTACAAAGGCAAGTACATTGACAAGAAGCAAGAAGATTTCGTCCCTAGCAACGCCTTACTTAGCCAGGACCGTCTTTGGCAGGCAGTAGAAAACTTGACGCAGTCTAATGAAACGATTGTTGCCGAACAGGGTACCTCCTTTTTCGGGGCGAGTAGTATTTTTTTGAAACCAAAGAGCCACTTTATCGGGCAACCATTATGGGGGTCCATTGGGTATACCTTCCCAGCTGCTTTAGGTTCCCAAATTGCAGACAAGGAGAGTCGTCATTTGCTGTTCATTGGTGATGGTTCATTGCAACTTACAGTGCAGGAACTGGGTTTAGCGATCCGTGAAAAAATTAACCCCATCTGCTTTATTATCAATAACGATGGTTATACCGTAGAGCGTGAAATTCATGGACCTAATCAATCGTATAATGATATTCCAATGTGGAACTACAGTAAGTTGCCTGAATCATTCGGCGCGACAGAGGAGCGCGTGGTCTCCAAAATCGTGCGCACAGAGAACGAATTCGTATCAGTGATGAAAGAAGCACAGGCCGACCCCAATCGCATGTATTGGATTGAACTGGTATTGGCGAAGGAAGACGCGCCTAAGGTACTGAAGAAGATGGGAAAACTTTTTGCTGAGCAAAACAAATCATAA

SEQ ID NO: 9 - protein, 1647 aa, Artificial Sequence
  KDC2 - mutant S286V
ATGTATACGGTGGGCGACTACCTGCTGGACCGTCTGCATGAGTTGGGAATTGAGGAAATCTTCGGCGTTCCCGGGGACTACAACTTGCAGTTTCTGGATCAAATCATTAGCCGCAAAGATATGAAATGGGTCGGCAATGCTAACGAGCTTAATGCAAGTTACATGGCCGACGGATATGCGCGTACTAAAAAAGCCGCAGCTTTCTTAACGACTTTCGGTGTGGGTGAACTTTCTGCAGTAAATGGGTTAGCTGGTTCATACGCCGAAAACTTACCAGTGGTGGAAATCGTCGGGTCGCCTACCTCAAAGGTCCAGAATGAAGGAAAATTTGTTCATCACACGCTGGCGGATGGAGACTTTAAGCATTTTATGAAAATGCACGAACCCGTTACAGCTGCCCGCACATTGCTGACAGCTGAAAACGCGACAGTGGAGATTGACCGTGTACTGAGTGCGCTGCTGAAGGAGCGCAAGCCTGTGTATATCAATCTGCCTGTCGACGTAGCAGCAGCGAAAGCTGAAAAGCCCTCATTACCACTGAAAAAGGAGAATCCCACTTCTAACACTAGCGACCAGGAAATCCTGAACAAAATCCAAGAATCGCTTAAGAACGCTAAAAAACCCATTGTCATTACAGGGCATGAGATTATTAGCTTTGGGCTTGAGAACACTGTAACTCAGTTTATTAGTAAGACGAAGTTACCCATCACAACCTTGAATTTTGGGAAGTCCTCCGTAGATGAAACCTTGCCCTCGTTCCTTGGTATCTACAACGGTAAACTGTCAGAGCCAAACCTTAAAGAGTTTGTGGAGTCGGCCGACTTCATTTTGATGTTGGGCGTGAAGCTGACTGACGTGTCTACAGGCGCGTTCACTCACCATTTAAATGAAAACAAGATGATCTCCCTTAACATCGACGAGGGCAAAATTTTTAATGAGTCGATTCAGAACTTCGACTTCGAGTCGTTAATTTCGTCCTTACTGGATCTTTCTGGTATTGAGTACAAAGGCAAGTACATTGACAAGAAGCAAGAAGATTTCGTCCCTAGCAACGCCTTACTTAGCCAGGACCGTCTTTGGCAGGCAGTAGAAAACTTGACGCAGTCTAATGAAACGATTGTTGCCGAACAGGGTACCTCCTTTTTCGGGGCGAGTAGTATTTTTTTGAAACCAAAGAGCCACTTTATCGGGCAACCATTATGGGGGTCCATTGGGTATACCTTCCCAGCTGCTTTAGGTTCCCAAATTGCAGACAAGGAGAGTCGTCATTTGCTGTTCATTGGTGATGGTTCATTGCAACTTACAGTGCAGGAACTGGGTTTAGCGATCCGTGAAAAAATTAACCCCATCTGCTTTATTATCAATAACGATGGTTATACCGTAGAGCGTGAAATTCATGGACCTAATCAATCGTATAATGATATTCCAATGTGGAACTACAGTAAGTTGCCTGAATCATTCGGCGCGACAGAGGAGCGCGTGGTCTCCAAAATCGTGCGCACAGAGAACGAATTCGTATCAGTGATGAAAGAAGCACAGGCCGACCCCAATCGCATGTATTGGATTGAACTGGTATTGGCGAAGGAAGACGCGCCTAAGGTACTGAAGAAGATGGGAAAACTTTTTGCTGAGCAAAACAAATCATAA

SEQ ID NO: 10 - protein, 1647 aa, Artificial Sequence
  KDC2 - mutant F110A
ATGTATACGGTGGGCGACTACCTGCTGGACCGTCTGCATGAGTTGGGAATTGAGGAAATCTTCGGCGTTCCCGGGGACTACAACTTGCAGTTTCTGGATCAAATCATTAGCCGCAAAGATATGAAATGGGTCGGCAATGCTAACGAGCTTAATGCAAGTTACATGGCCGACGGATATGCGCGTACTAAAAAAGCCGCAGCTTTCTTAACGACTTTCGGTGTGGGTGAACTTTCTGCAGTAAATGGGTTAGCTGGTTCATACGCCGAAAACTTACCAGTGGTGGAAATCGTCGGGTCGCCTACCTCAAAGGTCCAGAATGAAGGAAAAGCGGTTCATCACACGCTGGCGGATGGAGACTTTAAGCATTTTATGAAAATGCACGAACCCGTTACAGCTGCCCGCACATTGCTGACAGCTGAAAACGCGACAGTGGAGATTGACCGTGTACTGAGTGCGCTGCTGAAGGAGCGCAAGCCTGTGTATATCAATCTGCCTGTCGACGTAGCAGCAGCGAAAGCTGAAAAGCCCTCATTACCACTGAAAAAGGAGAATCCCACTTCTAACACTAGCGACCAGGAAATCCTGAACAAAATCCAAGAATCGCTTAAGAACGCTAAAAAACCCATTGTCATTACAGGGCATGAGATTATTAGCTTTGGGCTTGAGAACACTGTAACTCAGTTTATTAGTAAGACGAAGTTACCCATCACAACCTTGAATTTTGGGAAGTCCTCCGTAGATGAAACCTTGCCCTCGTTCCTTGGTATCTACAACGGTAAACTGTCAGAGCCAAACCTTAAAGAGTTTGTGGAGTCGGCCGACTTCATTTTGATGTTGGGCGTGAAGCTGACTGACTCGTCTACAGGCGCGTTCACTCACCATTTAAATGAAAACAAGATGATCTCCCTTAACATCGACGAGGGCAAAATTTTTAATGAGTCGATTCAGAACTTCGACTTCGAGTCGTTAATTTCGTCCTTACTGGATCTTTCTGGTATTGAGTACAAAGGCAAGTACATTGACAAGAAGCAAGAAGATTTCGTCCCTAGCAACGCCTTACTTAGCCAGGACCGTCTTTGGCAGGCAGTAGAAAACTTGACGCAGTCTAATGAAACGATTGTTGCCGAACAGGGTACCTCCTTTTTCGGGGCGAGTAGTATTTTTTTGAAACCAAAGAGCCACTTTATCGGGCAACCATTATGGGGGTCCATTGGGTATACCTTCCCAGCTGCTTTAGGTTCCCAAATTGCAGACAAGGAGAGTCGTCATTTGCTGTTCATTGGTGATGGTTCATTGCAACTTACAGTGCAGGAACTGGGTTTAGCGATCCGTGAAAAAATTAACCCCATCTGCTTTATTATCAATAACGATGGTTATACCGTAGAGCGTGAAATTCATGGACCTAATCAATCGTATAATGATATTCCAATGTGGAACTACAGTAAGTTGCCTGAATCATTCGGCGCGACAGAGGAGCGCGTGGTCTCCAAAATCGTGCGCACAGAGAACGAATTCGTATCAGTGATGAAAGAAGCACAGGCCGACCCCAATCGCATGTATTGGATTGAACTGGTATTGGCGAAGGAAGACGCGCCTAAGGTACTGAAGAAGATGGGAAAACTTTTTGCTGAGCAAAACAAATCATAA

SEQ ID NO: 11 - protein, 1647 aa, Artificial Sequence
  KDC2 - mutant G402A
ATGTATACGGTGGGCGACTACCTGCTGGACCGTCTGCATGAGTTGGGAATTGAGGAAATCTTCGGCGTTCCCGGGGACTACAACTTGCAGTTTCTGGATCAAATCATTAGCCGCAAAGATATGAAATGGGTCGGCAATGCTAACGAGCTTAATGCAAGTTACATGGCCGACGGATATGCGCGTACTAAAAAAGCCGCAGCTTTCTTAACGACTTTCGGTGTGGGTGAACTTTCTGCAGTAAATGGGTTAGCTGGTTCATACGCCGAAAACTTACCAGTGGTGGAAATCGTCGGGTCGCCTACCTCAAAGGTCCAGAATGAAGGAAAATTTGTTCATCACACGCTGGCGGATGGAGACTTTAAGCATTTTATGAAAATGCACGAACCCGTTACAGCTGCCCGCACATTGCTGACAGCTGAAAACGCGACAGTGGAGATTGACCGTGTACTGAGTGCGCTGCTGAAGGAGCGCAAGCCTGTGTATATCAATCTGCCTGTCGACGTAGCAGCAGCGAAAGCTGAAAAGCCCTCATTACCACTGAAAAAGGAGAATCCCACTTCTAACACTAGCGACCAGGAAATCCTGAACAAAATCCAAGAATCGCTTAAGAACGCTAAAAAACCCATTGTCATTACAGGGCATGAGATTATTAGCTTTGGGCTTGAGAACACTGTAACTCAGTTTATTAGTAAGACGAAGTTACCCATCACAACCTTGAATTTTGGGAAGTCCTCCGTAGATGAAACCTTGCCCTCGTTCCTTGGTATCTACAACGGTAAACTGTCAGAGCCAAACCTTAAAGAGTTTGTGGAGTCGGCCGACTTCATTTTGATGTTGGGCGTGAAGCTGACTGACTCGTCTACAGGCGCGTTCACTCACCATTTAAATGAAAACAAGATGATCTCCCTTAACATCGACGAGGGCAAAATTTTTAATGAGTCGATTCAGAACTTCGACTTCGAGTCGTTAATTTCGTCCTTACTGGATCTTTCTGGTATTGAGTACAAAGGCAAGTACATTGACAAGAAGCAAGAAGATTTCGTCCCTAGCAACGCCTTACTTAGCCAGGACCGTCTTTGGCAGGCAGTAGAAAACTTGACGCAGTCTAATGAAACGATTGTTGCCGAACAGGGTACCTCCTTTTTCGGGGCGAGTAGTATTTTTTTGAAACCAAAGAGCCACTTTATCGGGCAACCATTATGGGCGTCCATTGGGTATACCTTCCCAGCTGCTTTAGGTTCCCAAATTGCAGACAAGGAGAGTCGTCATTTGCTGTTCATTGGTGATGGTTCATTGCAACTTACAGTGCAGGAACTGGGTTTAGCGATCCGTGAAAAAATTAACCCCATCTGCTTTATTATCAATAACGATGGTTATACCGTAGAGCGTGAAATTCATGGACCTAATCAATCGTATAATGATATTCCAATGTGGAACTACAGTAAGTTGCCTGAATCATTCGGCGCGACAGAGGAGCGCGTGGTCTCCAAAATCGTGCGCACAGAGAACGAATTCGTATCAGTGATGAAAGAAGCACAGGCCGACCCCAATCGCATGTATTGGATTGAACTGGTATTGGCGAAGGAAGACGCGCCTAAGGTACTGAAGAAGATGGGAAAACTTTTTGCTGAGCAAAACAAATCATAA

SEQ ID NO: 12 - protein, 1647 aa, Artificial Sequence
  KDC2 - mutant F542L
ATGTATACGGTGGGCGACTACCTGCTGGACCGTCTGCATGAGTTGGGAATTGAGGAAATCTTCGGCGTTCCCGGGGACTACAACTTGCAGTTTCTGGATCAAATCATTAGCCGCAAAGATATGAAATGGGTCGGCAATGCTAACGAGCTTAATGCAAGTTACATGGCCGACGGATATGCGCGTACTAAAAAAGCCGCAGCTTTCTTAACGACTTTCGGTGTGGGTGAACTTTCTGCAGTAAATGGGTTAGCTGGTTCATACGCCGAAAACTTACCAGTGGTGGAAATCGTCGGGTCGCCTACCTCAAAGGTCCAGAATGAAGGAAAATTTGTTCATCACACGCTGGCGGATGGAGACTTTAAGCATTTTATGAAAATGCACGAACCCGTTACAGCTGCCCGCACATTGCTGACAGCTGAAAACGCGACAGTGGAGATTGACCGTGTACTGAGTGCGCTGCTGAAGGAGCGCAAGCCTGTGTATATCAATCTGCCTGTCGACGTAGCAGCAGCGAAAGCTGAAAAGCCCTCATTACCACTGAAAAAGGAGAATCCCACTTCTAACACTAGCGACCAGGAAATCCTGAACAAAATCCAAGAATCGCTTAAGAACGCTAAAAAACCCATTGTCATTACAGGGCATGAGATTATTAGCTTTGGGCTTGAGAACACTGTAACTCAGTTTATTAGTAAGACGAAGTTACCCATCACAACCTTGAATTTTGGGAAGTCCTCCGTAGATGAAACCTTGCCCTCGTTCCTTGGTATCTACAACGGTAAACTGTCAGAGCCAAACCTTAAAGAGTTTGTGGAGTCGGCCGACTTCATTTTGATGTTGGGCGTGAAGCTGACTGACTCGTCTACAGGCGCGTTCACTCACCATTTAAATGAAAACAAGATGATCTCCCTTAACATCGACGAGGGCAAAATTTTTAATGAGTCGATTCAGAACTTCGACTTCGAGTCGTTAATTTCGTCCTTACTGGATCTTTCTGGTATTGAGTACAAAGGCAAGTACATTGACAAGAAGCAAGAAGATTTCGTCCCTAGCAACGCCTTACTTAGCCAGGACCGTCTTTGGCAGGCAGTAGAAAACTTGACGCAGTCTAATGAAACGATTGTTGCCGAACAGGGTACCTCCTTTTTCGGGGCGAGTAGTATTTTTTTGAAACCAAAGAGCCACTTTATCGGGCAACCATTATGGGGGTCCATTGGGTATACCTTCCCAGCTGCTTTAGGTTCCCAAATTGCAGACAAGGAGAGTCGTCATTTGCTGTTCATTGGTGATGGTTCATTGCAACTTACAGTGCAGGAACTGGGTTTAGCGATCCGTGAAAAAATTAACCCCATCTGCTTTATTATCAATAACGATGGTTATACCGTAGAGCGTGAAATTCATGGACCTAATCAATCGTATAATGATATTCCAATGTGGAACTACAGTAAGTTGCCTGAATCATTCGGCGCGACAGAGGAGCGCGTGGTCTCCAAAATCGTGCGCACAGAGAACGAATTCGTATCAGTGATGAAAGAAGCACAGGCCGACCCCAATCGCATGTATTGGATTGAACTGGTATTGGCGAAGGAAGACGCGCCTAAGGTACTGAAGAAGATGGGAAAACTTCTGGCTGAGCAAAACAAATCATAA

SEQ ID NO: 13 - protein, 1707 aa, Artificial Sequence
  KDC3 - mutant T388A, I472V
ATGAGCTATACGGTAGGTACTTATTTGGCGGAGCGCCTTGTCCAGATCGGGCTTAAGCACCATTTTGCTGTGGCCGGTGATTACAACTTGGTTTTACTTGACAATCTGCTGTTAAATAAAAACATGGAGCAAGTGTACTGTTGTAATGAGCTGAATTGCGGATTCTCAGCGGAAGGGTATGCCCGTGCTAAAGGGGCCGCCGCTGCGGTTGTAACTTACAGTGTCGGCGCCTTAAGCGCCTTCGATGCGATTGGTGGGGCGTATGCCGAAAATCTTCCTGTCATCTTAATCTCCGGAGCACCAAATAATAATGATCACGCAGCAGGGCATGTGCTTCATCATGCTCTGGGAAAGACTGACTATCATTACCAGTTAGAGATGGCCAAGAACATTACGGCAGCCGCTGAGGCCATTTATACACCTGAAGAAGCTCCGGCTAAGATCGACCACGTAATTAAAACTGCCTTGCGCGAGAAAAAACCTGTGTACTTGGAAATCGCCTGTAATATTGCATCGATGCCATGTGCAGCACCAGGGCCTGCTTCAGCACTTTTTAATGACGAGGCTTCGGATGAGGCCAGCTTAAACGCAGCAGTCGAAGAGACGCTGAAGTTTATCGCGAATCGCGACAAGGTCGCTGTCCTGGTGGGTAGCAAACTGCGTGCAGCTGGTGCAGAGGAGGCGGCCGTTAAATTCGCTGATGCTTTGGGCGGGGCCGTAGCTACCATGGCGGCGGCTAAGAGTTTCTTTCCCGAAGAAAATCCCCACTACATCGGGACTAGTTGGGGTGAGGTCAGCTACCCTGGGGTCGAGAAAACAATGAAAGAAGCTGACGCGGTAATCGCCCTGGCTCCAGTATTCAACGACTACAGCACGACCGGGTGGACAGACATCCCAGATCCGAAGAAGTTAGTCTTAGCAGAACCTCGCAGCGTAGTCGTGAATGGCATCCGTTTTCCCTCAGTTCACCTTAAGGATTATCTTACCCGCTTGGCGCAGAAAGTCTCGAAGAAGACGGGCGCGTTGGATTTTTTCAAATCCCTTAACGCGGGCGAATTGAAGAAGGCTGCTCCCGCAGATCCCTCTGCTCCGTTGGTGAATGCCGAAATTGCACGTCAGGTGGAGGCTTTATTAACCCCAAATACGACAGTGATTGCAGAAGCGGGTGACTCATGGTTTAACGCCCAGCGTATGAAGTTGCCGAACGGAGCACGCGTGGAGTATGAAATGCAATGGGGACACATCGGTTGGTCCGTACCGGCAGCATTTGGATATGCTGTCGGCGCTCCGGAACGCCGTAACATTCTGATGGTTGGAGACGGGAGCTTTCAACTGACAGCGCAGGAGGTAGCACAAATGGTCCGCTTGAAACTGCCAGTCATCATCTTCCTGATTAACAATTATGGATACACTGTGGAAGTAATGATCCATGACGGTCCTTACAACAACATCAAAAATTGGGATTATGCTGGATTGATGGAGGTTTTCAACGGCAACGGGGGGTATGACTCGGGCGCGGGTAAAGGCTTGAAAGCTAAGACCGGCGGTGAGCTTGCAGAGGCGATCAAGGTCGCTCTTGCTAATACTGATGGTCCTACGTTGATTGAATGCTTTATCGGTCGCGAAGACTGTACTGAGGAATTGGTCAAGTGGGGTAAGCGCGTCGCCGCCGCGAACTCACGCAAGCCTGTAAACAAGTTACTGTAG

SEQ ID NO: 14 - protein, 1707 aa, Artificial Sequence
  KDC3 - mutant Y290F, T388S, I472V
ATGAGCTATACGGTAGGTACTTATTTGGCGGAGCGCCTTGTCCAGATCGGGCTTAAGCACCATTTTGCTGTGGCCGGTGATTACAACTTGGTTTTACTTGACAATCTGCTGTTAAATAAAAACATGGAGCAAGTGTACTGTTGTAATGAGCTGAATTGCGGATTCTCAGCGGAAGGGTATGCCCGTGCTAAAGGGGCCGCCGCTGCGGTTGTAACTTACAGTGTCGGCGCCTTAAGCGCCTTCGATGCGATTGGTGGGGCGTATGCCGAAAATCTTCCTGTCATCTTAATCTCCGGAGCACCAAATAATAATGATCACGCAGCAGGGCATGTGCTTCATCATGCTCTGGGAAAGACTGACTATCATTACCAGTTAGAGATGGCCAAGAACATTACGGCAGCCGCTGAGGCCATTTATACACCTGAAGAAGCTCCGGCTAAGATCGACCACGTAATTAAAACTGCCTTGCGCGAGAAAAAACCTGTGTACTTGGAAATCGCCTGTAATATTGCATCGATGCCATGTGCAGCACCAGGGCCTGCTTCAGCACTTTTTAATGACGAGGCTTCGGATGAGGCCAGCTTAAACGCAGCAGTCGAAGAGACGCTGAAGTTTATCGCGAATCGCGACAAGGTCGCTGTCCTGGTGGGTAGCAAACTGCGTGCAGCTGGTGCAGAGGAGGCGGCCGTTAAATTCGCTGATGCTTTGGGCGGGGCCGTAGCTACCATGGCGGCGGCTAAGAGTTTCTTTCCCGAAGAAAATCCCCACTACATCGGGACTAGTTGGGGTGAGGTCAGCTACCCTGGGGTCGAGAAAACAATGAAAGAAGCTGACGCGGTAATCGCCCTGGCTCCAGTATTCAACGACTTTAGCACGACCGGGTGGACAGACATCCCAGATCCGAAGAAGTTAGTCTTAGCAGAACCTCGCAGCGTAGTCGTGAATGGCATCCGTTTTCCCTCAGTTCACCTTAAGGATTATCTTACCCGCTTGGCGCAGAAAGTCTCGAAGAAGACGGGCGCGTTGGATTTTTTCAAATCCCTTAACGCGGGCGAATTGAAGAAGGCTGCTCCCGCAGATCCCTCTGCTCCGTTGGTGAATGCCGAAATTGCACGTCAGGTGGAGGCTTTATTAACCCCAAATACGACAGTGATTGCAGAAAGCGGTGACTCATGGTTTAACGCCCAGCGTATGAAGTTGCCGAACGGAGCACGCGTGGAGTATGAAATGCAATGGGGACACATCGGTTGGTCCGTACCGGCAGCATTTGGATATGCTGTCGGCGCTCCGGAACGCCGTAACATTCTGATGGTTGGAGACGGGAGCTTTCAACTGACAGCGCAGGAGGTAGCACAAATGGTCCGCTTGAAACTGCCAGTCATCATCTTCCTGATTAACAATTATGGATACACTGTGGAAGTAATGATCCATGACGGTCCTTACAACAACATCAAAAATTGGGATTATGCTGGATTGATGGAGGTTTTCAACGGCAACGGGGGGTATGACTCGGGCGCGGGTAAAGGCTTGAAAGCTAAGACCGGCGGTGAGCTTGCAGAGGCGATCAAGGTCGCTCTTGCTAATACTGATGGTCCTACGTTGATTGAATGCTTTATCGGTCGCGAAGACTGTACTGAGGAATTGGTCAAGTGGGGTAAGCGCGTCGCCGCCGCGAACTCACGCAAGCCTGTAAACAAGTTACTGTAG

SEQ ID NO: 15 - protein, 340 aa, Artificial Sequence
  alcohol dehydrogenase 1
MKAAVVRHNPDGYADLVEKELRAIKPNEALLDMEYCGVCHTDLHVAAGDFGNKAGTVLGHEGIGIVKEIGADVSSLQVGDRVSVAWFFEGCGHCEYCVSGNETFCREVKSAGYSVDGGMAEEAIVVADYAVKVPDGLDPIEASSITCAGVTTYKAIKVSGVKPGDWQVIFGAGGLGNLAIQYAKNVFGAKVIAVDINQDKLNLAKKIGADVTINSGDVNPVDEIKKITGGLGVQSAIVCAVARIAFEQAVASLKPMGKMVAVAVPNTEMTLSVPTVVFDGVEVAGSLVGTRLDLAEAFQFGAEGKVKPIVATRKLEEINDIIDEMKAGKIEGRMVIDFTK

SEQ ID NO: 16 - protein, 375 aa, Unknown
  alcohol dehydrogenase 2
MSTAGKVIKCKAAVLWEEKKPFSIEEVEVAPPKAHEVRIKMVATGICRSDDHVVSGTLVTPLPVIAGHEAAGIVESIGEGVTTVRPGDKVIPLFTPQCGKCRVCKHPEGNFCLKNDLSMPRGTMQDGTSRFTCRGKPIHHFLGTSTFSQYTVVDEISVAKIDAASPLEKVCLIGCGFSTGYGSAVKVAKVTQGSTCAVFGLGGVGLSVIMGCKAAGAARIIGVDINKDKFAKAKEVGATECVNPQDYKKPIQEVLTEMSNGGVDFSFEVIGRLDTMVTALSCCQEAYGVSVIVGVPPDSQNLSMNPMLLLSGRTWKGAIFGGFKSKDSVPKLVADFMAKKFALDPLITHVLPFEKINEGFDLLRSGESIRTILTF

SEQ ID NO: 17 - protein, 499 aa, Unknown
  aldehyde dehydrogenase
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADHLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 18 - protein, 548 aa, Artificial Sequence
  KDC2 - mutant S286V
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENPTSNTSDQEILNKIQESLKNAKKPIVITGHEIISFGLENTVTQFISKTKLPITTLNFGKSSVDETLPSFLGIYNGKLSEPNLKEFVESADFILMLGVKLTDVSTGAFTHHLNENKMISLNIDEGKIFNESIQNFDFESLISSLLDLSGIEYKGKYIDKKQEDFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELVLAKEDAPKVLKKMGKLFAEQNKS

SEQ ID NO: 19 - protein, 548 aa, Artificial Sequence
  KDC2 - mutant F110A
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKAVHHTLADGDFKHFMKMHEPVTAARTLLTAENATVEIDRVLSALLKERKPVYINLPVDVAAAKAEKPSLPLKKENPTSNTSDQEILNKIQESLKNAKKPIVITGHEIISFGLENTVTQFISKTKLPITTLNFGKSSVDETLPSFLGIYNGKLSEPNLKEFVESADFILMLGVKLTDSSTGAFTHHLNENKMISLNIDEGKIFNESIQNFDFESLISSLLDLSGIEYKGKYIDKKQEDFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELVLAKEDAPKVLKKMGKLFAEQNKS

SEQ ID NO: 20 - protein, 548 aa, Artificial Sequence
  KDC2 - mutant F542L
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENPTSNTSDQEILNKIQESLKNAKKPIVITGHEIISFGLENTVTQFISKTKLPITTLNFGKSSVDETLPSFLGIYNGKLSEPNLKEFVESADFILMLGVKLTDSSTGAFTHHLNENKMISLNIDEGKIFNESIQNFDFESLISSLLDLSGIEYKGKYIDKKQEDFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELVLAKEDAPKVLKKMGKLLAEQNKS

SEQ ID NO: 21 - protein, 548 aa, Artificial Sequence
  KDC2 - mutant S286L
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENPTSNTSDQEILNKIQESLKNAKKPIVITGHEIISFGLENTVTQFISKTKLPITTLNFGKSSVDETLPSFLGIYNGKLSEPNLKEFVESADFILMLGVKLTDLSTGAFTHHLNENKMISLNIDEGKIFNESIQNFDFESLISSLLDLSGIEYKGKYIDKKQEDFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELVLAKEDAPKVLKKMGKLFAEQNKS

SEQ ID NO: 22 - protein, 548 aa, Artificial Sequence
  KDC2 - mutant G402A
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENPTSNTSDQEILNKIQESLKNAKKPIVITGHEIISFGLENTVTQFISKTKLPITTLNFGKSSVDETLPSFLGIYNGKLSEPNLKEFVESADFILMLGVKLTDSSTGAFTHHLNENKMISLNIDEGKIFNESIQNFDFESLISSLLDLSGIEYKGKYIDKKQEDFVPSNALLSQDRLWQAVENLTQSNETIVAEQGTSFFGASSIFLKPKSHFIGQPLWASIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELVLAKEDAPKVLKKMGKLFAEQNKS

SEQ ID NO: 23 - DNA, 24 bp, Artificial Sequence
  KCD2 forward primer
atgtacactgttggtgattacttg

SEQ ID NO: 24 - DNA, 25 bp, Artificial Sequence
  KDC2 reverse primer
ttaagacttgttttgttcagcgaac